(19) United States
(12) Patent Application Publication
(10) Pub. No.: US 2022/0168145 A1
(43) Pub. Date: Jun. 2, 2022

(54) TOOLS AND METHODS FOR DACRYOCYSTORHINOSTOMY

(71) Applicant: TEARFLOW CARE LTD., Haifa (IL)

(72) Inventors: Arie NEMET, Givat Shmuel (IL); Idan TOBIS, Beth Hashmonai (IL); Nir LILACH, Kfar Yehoshua (IL); Eliahu ELIACHAR, Haifa (IL); Assaf AGOU, Haifa (IL); Refael ISRAELI, Haifa (IL)

(21) Appl. No.: 17/601,067

(22) PCT Filed: Apr. 2, 2020
(86) PCT No.: PCT/IL2020/050399

§ 371 Date: Oct. 3, 2021

Related U.S. Application Data
(60) Provisional application No. 62/828,656, filed on Apr. 3, 2019.

(51) Int. Cl. A61F 9/007; A61B 17/17; A61B 17/16; A61F 9/008

(52) U.S. Cl. CPC A61F 9/00772; A61B 17/1785; A61F 9/008; A61B 17/1615; A61B 17/1688

(57) ABSTRACT

Embodiments of the present invention provide methods for performing dacryocystorhinostomy and an apparatus for performing dacryocystorhinostomy (DCR), the apparatus comprising a dacryocystorhinostomy (DCR) tool, which includes a perforating shaft having a distal perforating-shaft perforating tip configured to form a bypass between a lacrimal sac and a nasal cavity through a lateral side of the lacrimal sac, a lacrimal bone, and nasal mucosa and a DCR guide, which includes a nasal guide component, which is configured to be inserted into the nasal cavity and has a nasal-guide channel having a proximal opening and an at least partially laterally facing distal opening, and a lacrimal guide component, which is shaped so as to define a lacrimal-guide channel that is configured to orient the distal perforating-shaft perforating tip with respect to the DCR guide during advancing of the distal perforating-shaft perforating tip through a lacrimal passageway and into the lacrimal sac, until the distal perforating-shaft perforating tip at least crosses the laterally facing distal opening of the nasal guide component, and wherein the DCR guide is configured to constrain the laterally facing distal opening of the nasal guide component to fall in a path of advancement of the distal perforating-shaft perforating tip.

### CROSS-REFERENCE TO RELATED APPLICATIONS

This application claims the benefit of priority from U.S. Provisional Patent Application No. 62/828,656, filed on Apr. 3, 2019, entitled “TOOLS AND METHODS FOR DACRYOCYSTORHINOSTOMY”.

This application is related to PCT Patent Application No. PCT/IL2018/050943, filed on Aug. 27, 2018, entitled “TOOLS AND METHODS FOR DACRYOCYSTORHINOSTOMY”, which claims the benefit of priority from U.S. Provisional Patent Application No. 62/563,812, filed on Sep. 27, 2017.

The contents of the above application are incorporated by reference herein in their entirety.

### FIELD OF THE APPLICATION

The present invention relates generally to surgical eye procedures, and specifically to tools and methods for performing dacryocystorhinostomy.

### BACKGROUND OF THE APPLICATION

Blockage of the nasolacrimal (tear) duct in adults results in excessive tearing (epiphora), which causes suffering, a substantial disruption to the patient's ability to function, and a substantial reduction in quality of life. Blockage can cause severe infections of the nasolacrimal ducts and the eye socket, and danger to the eye.

Dacryocystorhinostomy (DCR) is a surgical procedure for restoring the flow of tears into the nose from the lacrimal sac when the nasolacrimal duct is blocked, by opening the blockage and forming a bypass for drainage toward the nose. An external approach to DCR was developed in 1904 and includes cutting skin, muscle, bone, and nasal mucosa and bypass formation. DCR can also be performed endoscopically through the nose, to form a bypass between the nasolacrimal duct and the nose. It is estimated that about 40% of the DCR surgeries are performed using the endoscopic method and about 60% using the external approach. The two operations are similarly complicated and require special training. This surgery may be performed by specialists in oculoplasty or otorhinolaryngology, but surgery is often performed by a team of two specials from both fields. In general, DCR is a complex surgery that is usually performed under general anesthesia or deep blur, and which may involve scars and discomfort. DCR success rates are generally 75-90%. Therefore, many patients and surgeons prefer to avoid treating epiphora, and patients continue to suffer.

### SUMMARY OF THE APPLICATION

Embodiments of the present invention provide dacryocystorhinostomy (DCR) tools and methods for performing dacryocystorhinostomy. The DCR tools comprise a DCR guide and, for some applications, a perforating shaft having a distal perforating-shaft perforating tip configured to form a bypass between a lacrimal sac and a nasal cavity through a lateral side of the lacrimal sac, a lacrimal bone, and nasal mucosa.

The DCR guide comprises a nasal guide component and a lacrimal guide component. The nasal guide component is configured to be inserted into the nasal cavity and has a laterally facing distal opening. The lacrimal guide component is shaped so as to define a lacrimal-guide channel that is configured to orient the DCR guide (via the lacrimal guide component) with respect to guide the distal perforating-shaft perforating tip during advancing of the distal perforating-shaft perforating tip through a lacrimal passageway and into a lacrimal sac, until contact of the distal perforating-shaft perforating tip with an internal wall of the nasal-shaft-accepting channel blocks further advancing of distal perforating-shaft perforating tip of nasal guide component.

The DCR guide is configured to constrain the laterally facing distal opening of the nasal guide component to fall in a path of advancement of the distal perforating-shaft perforating tip. The lacrimal passageway to a large extent sets the path of advancement of the distal perforating-shaft perforating tip, which in turn sets an orientation and location of the perforating shaft. The perforating shaft in turn sets an orientation and location of the lacrimal guide component, which sets an orientation and location of the nasal guide component, including the laterally facing distal opening, in the nasal cavity. As a result, the laterally facing distal opening is automatically and non-electrically positioned in the path of advancement of the distal perforating-shaft perforating tip, and thus comes in contact with the internal wall of the nasal-shaft-accepting channel and blocks its advancement.

For some applications, the nasal guide component, including the laterally facing distal opening, is shaped so as to define a nasal-guide channel, which is configured to be guidewire-accepting. Typically, a distal opening of the nasal-guide channel faces at least partially in a lateral direction that faces toward the lacrimal guide component.

For some applications, the DCR tool further comprises, in addition to the perforating shaft, a lacrimal guidewire shaft having a distal tip. In these applications, the perforating shaft is typically solid, i.e., does not define a channel therethrough. The lacrimal guidewire shaft, including the distal tip thereof, is shaped so as to define a lacrimal guidewire-accepting channel. The lacrimal-guide channel of the lacrimal guide component is configured to orient the DCR guide (via the lacrimal guide component) with respect to the distal tip of the lacrimal guidewire shaft during advancing of the distal tip of the lacrimal guidewire shaft through the lacrimal-guide channel and the lacrimal passageway and into the lacrimal sac. The DCR guide is configured to constrain the laterally facing distal opening of the nasal guide component to fall in a path of advancement of the distal tip of the lacrimal guidewire shaft.

For other applications, the perforating shaft, including the distal perforating-shaft perforating tip, is shaped so as to define the lacrimal guidewire-accepting channel.

For some applications, the nasal guide component, including the laterally facing distal opening, is shaped so as to define a nasal-guide channel, which is shaped so as to define proximal and distal openings and configured to be nasal-perforating-tool-accepting. The DCR tool further comprises a nasal perforating tool, which is sized to pass through the nasal-guide channel, including the proximal and the distal openings thereof. The nasal perforating tool comprises a distal nasal-tool perforating tip configured to widen the bypass between the lacrimal sac and the nasal cavity, a proximal elongate shaft, and a distal flexible portion disposed longitudinally between the proximal elongate shaft and the distal nasal-tool perforating tip.

For some applications, a method of performing DCR is provided. The method does not require making an incision through skin, since access to the bypass is provided directly through the natural lacrimal punctum and canaliculi. The nasal guide component of the DCR guide is inserted into a nasal cavity of a patient's body. The nasal guide component need not be inserted precisely by the surgeon, so long as it is inserted into the correct nostril, because it will be precisely oriented and positioned by the DCR guide, as described hereinbelow. The perforating shaft is advanced through the lacrimal-guide channel of the lacrimal guide component of the DCR guide and a lacrimal passageway and into a lacrimal sac. The lacrimal passageway includes a lacrimal punctum (either inferior or superior), a lacrimal canal (either inferior or superior), and a common canaliculus. Surgeons skilled in the DCR art generally are able to advance the perforating shaft through the lacrimal passageway without difficulty.

The DCR guide constrains the laterally facing distal opening of the nasal guide component to fall in a path of advancement of the distal perforating-shaft perforating tip. As a result of this constraint, the DCR guide typically positions the laterally facing distal opening of the nasal guide component at an axilla of a middle turbinate of the nasal cavity. A bypass is formed between the lacrimal sac and the nasal cavity by advancing the distal perforating-shaft perforating tip through a lateral side of the lacrimal sac, a lacrimal bone, and nasal mucosa, until contact of the distal perforating-shaft perforating tip with the internal wall of the nasal-shaft-accepting channel of the nasal guide component blocks further advancing of the distal perforating-shaft perforating tip. This contact prevents over-advancement the distal perforating-shaft perforating tip, which might otherwise perforate tissue across the nasal cavity, which is generally no more than several millimeters beyond the bypass.

For some applications, a guidewire is placed such that the guidewire passes through the lacrimal passageway, the bypass, and at least a portion of the nasal-guide channel. Typically, the guidewire is placed such that the guidewire passes through the lacrimal passageway, the bypass, the entire the nasal-guide channel, and out of a proximal end of the nasal-guide channel. Typically, the distal opening of the nasal-guide channel faces at least partially in the above-mentioned lateral direction that faces toward the lacrimal guide component.

For some applications, a dilator is advanced along and over the guidewire and through the lacrimal passageway and into the bypass, and the bypass is dilated using the dilator. For some applications, a tubular support element is advanced along and over the guidewire and through the lacrimal passageway and into the bypass, and the guidewire is removed from the patient's body while leaving the tubular support element in place in the bypass.

For some applications, the nasal guide component, including the laterally facing distal opening, is shaped so as to define a nasal-guide channel, which is shaped so as to define proximal and distal openings and is configured to be nasal-perforating-tool-accepting. The method further comprises inserting the above-described nasal perforating tool into the proximal opening of the nasal-guide channel. The nasal perforating tool is advanced within the nasal-guide channel until the distal nasal-tool perforating tip exits the distal opening of the distal nasal-tool perforating tip as the distal flexible portion flexes. The distal nasal-tool perforating tip is used to widen the bypass between the lacrimal sac and the nasal cavity.

There is therefore provided, in accordance with an application of the present invention, apparatus for performing dacryocystorhinostomy (DCR), the apparatus including a dacryocystorhinostomy (DCR) tool, which includes:

a perforating shaft having a distal perforating-shaft perforating tip configured to form a bypass between a lacrimal sac and a nasal cavity through a lateral side of the lacrimal sac, a lacrimal bone, and nasal mucosa; and

a DCR guide, which includes:

a nasal guide component, which is configured to be inserted into the nasal cavity and has a laterally facing distal opening; and

a lacrimal guide component, which is shaped so as to define a lacrimal-guide channel that is configured to orient the DCR guide with respect to the distal perforating-shaft perforating tip during advancing of the distal perforating-shaft perforating tip through a lacrimal passageway and into the lacrimal sac, until contact of the distal perforating-shaft perforating tip with the internal wall of the nasal-shaft-accepting channel of the nasal guide component blocks further advancing of the distal perforating-shaft perforating tip, the lacrimal passageway including a lacrimal punctum, a lacrimal canal, and a common canaliculus,

wherein the DCR guide is configured to constrain the laterally facing distal opening of the nasal guide component to fall in a path of advancement of the distal perforating-shaft perforating tip.

For some applications, the DCR guide is configured to set a desired angle between respective central longitudinal axes of the nasal guide component and the perforating shaft.

For some applications, the DCR guide is shaped so as to define an arcuate portion that is configured to allow relative movement between the nasal guide component and the lacrimal guide component to set the desired angle.

For some applications, the distal perforating-shaft perforating tip is shaped as a drill bit.

For some applications, the distal perforating-shaft perforating tip is shaped as a punch.

For some applications, the nasal guide component, including the laterally facing distal opening, is shaped so as to define a nasal-guide channel, which is configured to be guidewire-accepting.

For some applications, a distal opening of the nasal-guide channel faces at least partially in a lateral direction that faces toward the lacrimal guide component.

For some applications:

the nasal guide component includes (a) an outer guide element that is shaped so as to define a nasal-shaft-accepting channel therethrough, and (b) a nasal shaft that is slidable through the nasal-shaft-accepting channel, and the nasal shaft is shaped so as to define the laterally facing distal opening and the nasal-guide channel, and

when the outer guide element and the nasal shaft are coupled together, a distal opening of the nasal-guide channel is constrained by the outer guide element and the nasal shaft to face at least partially in a lateral direction that faces toward the lacrimal guide component.

For some applications, the nasal guide component includes a locking mechanism, which is configured to lock the nasal shaft rotationally with respect to the nasal-shaft-accepting channel, thereby maintaining the distal opening of the nasal-guide channel facing at least partially in the lateral direction.

For some applications, the perforating shaft, including the distal perforating-shaft perforating tip, is shaped so as to define a lacrimal guidewire-accepting channel.

For some applications:

the DCR tool further includes a lacrimal guidewire shaft having a distal tip, and the lacrimal guidewire shaft, including the distal tip thereof, is shaped so as to define a lacrimal guidewire-accepting channel,

the lacrimal-guide channel of the lacrimal guide component is configured to orient the DCR guide with respect to the distal tip of the lacrimal guidewire shaft during advancing of the distal tip through the lacrimal-guide channel and the lacrimal passageway and into the lacrimal sac, and

the DCR guide is configured to constrain the laterally facing distal opening of the nasal guide component to fall in a path of advancement of the distal tip of the lacrimal guidewire shaft.

For some applications, the DCR tool further includes a dilator, which is configured to be advanced through the lacrimal passageway and into the bypass, and to dilate the bypass.

For some applications, the dilator includes an inflatable element, which is configured to dilate the bypass by being inflated in the bypass.

For some applications, the apparatus further includes a tubular support element, which is configured to be advanced through the lacrimal passageway and into the bypass, and to maintain patency of the bypass.

For some applications, the nasal guide component includes (a) an outer guide element that is shaped so as to define a nasal-shaft-accepting channel therethrough, and (b) a nasal shaft that is slidable through the nasal-shaft-accepting channel, and the nasal shaft is shaped so as to define the laterally facing distal opening.

For some applications:

the nasal guide component, including the laterally facing distal opening, is shaped so as to define a nasal-guide channel, which is shaped so as to define proximal and distal openings and configured to be nasal-perforating-tool-accepting, and

the DCR tool further includes a nasal perforating tool, which is sized to pass through the nasal-guide channel, including the proximal and the distal openings thereof, and which includes:

a distal nasal-tool perforating tip configured to widen the bypass between the lacrimal sac and the nasal cavity;

a proximal elongate shaft; and

a distal flexible portion disposed longitudinally between the proximal elongate shaft and the distal nasal-tool perforating tip.

For some applications, the distal nasal-tool perforating tip is shaped as a drill bit.

For some applications, the distal nasal-tool perforating tip is shaped as a punch.

For some applications, the distal flexible portion is sufficiently flexible to allow flexing of a central longitudinal axis of the distal nasal-tool perforating tip by at least 15 degrees with respect to a central longitudinal axis of the nasal-guide channel when the distal nasal-tool perforating tip is advanced out of the distal opening of the nasal-guide channel while the nasal guide component is within the nasal cavity.

For some applications, the distal flexible portion is sufficiently flexible to allow flexing of the central longitudinal axis of the distal nasal-tool perforating tip by at least 40 degrees with respect to the central longitudinal axis of the nasal-guide channel when the distal nasal-tool perforating tip is advanced out of the distal opening of the nasal-guide channel while the nasal guide component is within the nasal cavity.

For some applications, the distal flexible portion is sufficiently flexible to allow flexing of a central longitudinal axis of the distal nasal-tool perforating tip by at least 15 degrees with respect to a central longitudinal axis of the nasal-guide channel without causing plastic deformation of the distal flexible portion.

For some applications, the distal flexible portion is sufficiently flexible to allow flexing of the central longitudinal axis of the distal nasal-tool perforating tip by at least 40 degrees with respect to the central longitudinal axis of the nasal-guide channel without causing plastic deformation of the distal flexible portion.

For some applications, the distal opening of the nasal-guide channel faces at least partially in a lateral direction that faces toward the lacrimal guide component.

For some applications, the nasal-guide channel is shaped so as to define a distal surface that faces partially in a proximal direction and partially in the lateral direction toward the lacrimal guide component so as to direct the distal nasal-tool perforating tip out of the distal opening of the nasal-guide channel, thereby flexing the distal flexible portion, when the nasal perforating tool is advanced distally through the nasal-guide channel.

For some applications, the distal flexible portion includes a coil.

For some applications, the nasal perforating tool is shaped so as to define a nasal-perforating-tool guidewire-accepting channel.

For some applications, the nasal perforating tool is shaped such that the nasal-perforating-tool guidewire-accepting channel has a distal opening at a distal end of the distal nasal-tool perforating tip.

For some applications, the dacryocystorhinostomy (DCR) tool device is for use with a guidewire, and the distal flexible portion is sufficiently flexible to allow flexing of a central longitudinal axis of the distal nasal-tool perforating tip by at least 15 degrees with respect to a central longitudinal axis of the nasal-guide channel when the distal nasal-tool perforating tip is advanced over the guidewire and out of the distal opening of the nasal-guide channel while the nasal guide component is within the nasal cavity and the guidewire is within the nasal-perforating-tool guidewire-accepting channel.

For some applications, the distal opening of the nasal-guide channel faces at least partially in a lateral direction that faces toward the lacrimal guide component.

For some applications, the nasal-guide channel is shaped so as to define a distal surface that faces partially in a proximal direction and partially in the lateral direction toward the lacrimal guide component so as to direct the distal nasal-tool perforating tip out of the distal opening of the nasal-guide channel, thereby flexing the distal flexible portion, when the nasal perforating tool is advanced distally through the nasal-guide channel.

There is further provided, in accordance with an application of the present invention, apparatus for performing dacryocystorhinostomy (DCR), the apparatus for use with a guidewire and including a dacryocystorhinostomy (DCR) tool, which includes:

a lacrimal guidewire shaft, which is configured to be inserted into a lacrimal passageway and has a distal tip, wherein the lacrimal guidewire shaft, including the distal tip, is shaped so as to define a lacrimal guidewire-accepting channel, and wherein the lacrimal passageway includes a lacrimal punctum, a lacrimal canal, and a common canaliculus; and

a DCR guide, which includes:

a nasal guide component, which is configured to be inserted into the nasal cavity and has a laterally facing distal opening, wherein the nasal guide component, including the laterally facing distal opening, is shaped so as to define a nasal-guide channel, which is configured to be guidewire-accepting; and

a lacrimal guide component, which is shaped so as to define a lacrimal-guide channel that is configured to orient the DCR guide with respect to the distal tip of the lacrimal guidewire shaft during advancing of the distal tip through the lacrimal passageway and into a lacrimal sac, until contact of the distal tip of the lacrimal guidewire shaft with the internal wall of the nasal-shaft-accepting channel of the nasal guide component blocks further advancing of the distal tip of the lacrimal guidewire shaft,

wherein the DCR guide is configured to constrain the laterally facing distal opening of the nasal guide component to fall in a path of advancement of the distal tip of the lacrimal guidewire shaft.

For some applications, the DCR guide is configured to set a desired angle between respective central longitudinal axes of the nasal guide component and the lacrimal guidewire shaft.

For some applications, the DCR guide is shaped so as to define an arcuate portion that is configured to allow relative movement between the nasal guide component and the lacrimal guide component to set the desired angle.

For some applications, a distal opening of the nasal-guide channel faces at least partially in a lateral direction that faces toward the lacrimal guide component.

For some applications:

the nasal guide component includes (a) an outer guide element that is shaped so as to define a nasal-shaft-accepting channel therethrough, and (b) a nasal shaft that is slidable through the nasal-shaft-accepting channel, and the nasal shaft is shaped so as to define the laterally facing distal opening and the nasal-guide channel, and

when the outer guide element and the nasal shaft are coupled together, a distal opening of the nasal-guide channel is constrained by the outer guide element and the nasal shaft to faces at least partially in a lateral direction that faces toward the lacrimal guide component.

For some applications, the nasal guide component includes a locking mechanism, which is configured to lock the nasal shaft rotationally with respect to the nasal-shaft-accepting channel, thereby maintaining the distal opening of the nasal-guide channel facing at least partially in the lateral direction.

There is still further provided, in accordance with an application of the present invention, a method of performing dacryocystorhinostomy (DCR), the method including:

inserting a nasal guide component of a dacryocystorhinostomy (DCR) guide into a nasal cavity of a patient's body;

advancing a perforating shaft through a lacrimal-guide channel of a lacrimal guide component of the DCR guide and a lacrimal passageway and into a lacrimal sac, the lacrimal passageway including a lacrimal punctum, a lacrimal canal, and a common canaliculus; and

forming a bypass between the lacrimal sac and the nasal cavity by advancing a distal perforating-shaft perforating tip through a lateral side of the lacrimal sac, a lacrimal bone, and nasal mucosa, until contact of the distal perforating-shaft perforating tip with an internal wall of the nasal-shaft-accepting channel of the nasal guide component blocks further advancing of the distal perforating-shaft perforating tip, wherein the DCR guide constrains the laterally facing distal opening of the nasal guide component to fall in a path of advancement of the distal perforating-shaft perforating tip.

For some applications, inserting the nasal guide component and the lacrimal guide component includes using the DCR guide to set a desired angle between respective central longitudinal axes of the nasal guide component and the perforating shaft.

For some applications, setting the desired angle includes using an arcuate portion of the DCR guide that allows relative movement between the nasal guide component and the lacrimal guide component to set the desired angle.

For some applications:

the nasal guide component, including the laterally facing distal opening, is shaped so as to define a nasal-guide channel, and

the method further includes, after forming the bypass, placing a guidewire such that the guidewire passes through the lacrimal passageway, the bypass, and at least a portion of the nasal-guide channel.

For some applications, placing the guidewire includes placing the guidewire such that the guidewire passes through the lacrimal passageway, the bypass, the entire nasal-guide channel, and out of a proximal end of the nasal-guide channel.

For some applications, a distal opening of the nasal-guide channel faces at least partially in a lateral direction that faces toward the lacrimal guide component.

For some applications:

the nasal guide component includes (a) an outer guide element that is shaped so as to define a nasal-shaft-accepting channel therethrough, and (b) a nasal shaft that is slidable through the nasal-shaft-accepting channel, and the nasal shaft is shaped so as to define the laterally facing distal opening and the nasal-guide channel,

the method further includes coupling together the outer guide element and the nasal shaft by sliding the nasal shaft through the nasal-shaft-accepting channel, and

when the outer guide element and the nasal shaft are coupled together, a distal opening of the nasal-guide channel is constrained by the outer guide element and the nasal shaft to face at least partially in a lateral direction that faces toward the lacrimal guide component.

For some applications, coupling together the outer guide element and the nasal shaft further includes locking the nasal shaft rotationally with respect to the nasal-shaft-accepting channel, thereby maintaining the distal opening of the nasal-guide channel facing at least partially in the lateral direction.

For some applications:

the perforating shaft, including the distal perforating-shaft perforating tip, is shaped so as to define a lacrimal guidewire-accepting channel, and

placing the guidewire such that the guidewire passes through the lacrimal passageway includes advancing the guidewire through the lacrimal guidewire-accepting channel while the perforating shaft is disposed passing through the lacrimal passageway.

For some applications, placing the guidewire such that the guidewire passes through the lacrimal passageway, the bypass, and the nasal-guide channel includes advancing the guidewire through the lacrimal guidewire-accepting channel while (a) the perforating shaft is disposed passing through the lacrimal passageway and the bypass, and (b) the distal perforating-shaft perforating tip is in contact with the laterally facing distal opening of the nasal guide component.

For some applications, placing the guidewire such that the guidewire passes through the lacrimal passageway includes:

removing the perforating shaft from the patient's body;

inserting a lacrimal guidewire shaft through the lacrimal-guide channel of the lacrimal guide component and the lacrimal passageway and into the lacrimal sac, wherein the lacrimal guidewire shaft, including a distal tip thereof, is shaped so as to define a lacrimal guidewire-accepting channel, and wherein the DCR guide constrains the laterally facing distal opening of the nasal guide component to fall in a path of advancement of the distal tip of the lacrimal guidewire shaft; and

advancing the guidewire through the lacrimal guidewire-accepting channel while the lacrimal guidewire shaft is disposed passing through the lacrimal passageway.

For some applications, placing the guidewire such that the guidewire passes through the lacrimal passageway, the bypass, and the nasal-guide channel includes advancing the guidewire through the lacrimal guidewire-accepting channel while (a) the lacrimal guidewire shaft is disposed passing through the lacrimal passageway and the bypass, and (b) the distal tip of the lacrimal guidewire shaft is in contact with the laterally facing distal opening of the nasal guide component.

For some applications, placing the guidewire includes placing the guidewire such that a first end of the guidewire extends out of the patient's body through the lacrimal punctum and a second end of the guidewire, opposite the first end, extends out of the patient's body through the nasal cavity.

For some applications, the method further includes, after placing the guidewire:

advancing a dilator along and over the guidewire and through the lacrimal passageway and into the bypass; and

dilating the bypass using the dilator.

For some applications, the dilator includes an inflatable element, and dilating the bypass includes inflating the inflatable element in the bypass.

For some applications, the method further includes, after dilating the bypass:

advancing a tubular support element along and over the guidewire and through the lacrimal passageway and into the bypass; and

removing the guidewire from the patient's body while leaving the tubular support element in place in the bypass.

For some applications:

the nasal guide component includes (a) an outer guide element that is shaped so as to define a nasal-shaft-accepting channel therethrough, and (b) a nasal shaft that is slidable through the nasal-shaft-accepting channel, and the nasal shaft is shaped so as to define the laterally facing distal opening, and

the method further includes coupling together the outer guide element and the nasal shaft by sliding the nasal shaft through the nasal-shaft-accepting channel.

For some applications, advancing the distal perforating-shaft perforating tip includes drilling the distal perforating-shaft perforating tip through the lateral side of the lacrimal sac, the lacrimal bone, and the nasal mucosa.

For some applications, advancing the distal perforating-shaft perforating tip includes punching the distal perforating-shaft perforating tip through the lateral side of the lacrimal sac, the lacrimal bone, and the nasal mucosa.

For some applications:

the nasal guide component, including the laterally facing distal opening, is shaped so as to define a nasal-guide channel, which is shaped so as to define proximal and distal openings and is configured to be nasal-perforating-tool-accepting, and

the method further includes:

inserting a nasal perforating tool into the proximal opening of the nasal-guide channel, the nasal perforating tool including a distal nasal-tool perforating tip, a proximal elongate shaft, and a distal flexible portion disposed longitudinally between the proximal elongate shaft and the distal nasal-tool perforating tip;

advancing the nasal perforating tool within the nasal-guide channel until the distal nasal-tool perforating tip exits the distal opening of the distal nasal-tool perforating tip as the distal flexible portion flexes; and

using the distal nasal-tool perforating tip, widening the bypass between the lacrimal sac and the nasal cavity.

For some applications, widening the bypass includes drilling using the distal nasal-tool perforating tip.

For some applications, widening the bypass includes punching using the distal nasal-tool perforating tip.

For some applications, advancing the nasal perforating tool within the nasal-guide channel until the distal nasal-tool perforating tip exits the distal opening of the distal nasal-tool perforating tip includes advancing the nasal perforating tool within the nasal-guide channel until the distal nasal-tool perforating tip exits the distal opening of the distal nasal-tool perforating tip as a central longitudinal axis of the distal nasal-tool perforating tip flexes by at least 15 degrees with respect to a central longitudinal axis of the nasal-guide channel.

For some applications, advancing the nasal perforating tool within the nasal-guide channel until the distal nasal-tool perforating tip exits the distal opening of the distal nasal-tool perforating tip includes advancing the nasal perforating tool within the nasal-guide channel until the distal nasal-tool perforating tip exits the distal opening of the distal nasal-tool perforating tip as the central longitudinal axis of the distal nasal-tool perforating tip flexes by at least 40 degrees with respect to the central longitudinal axis of the nasal-guide channel.

For some applications, the distal flexible portion is sufficiently flexible to allow flexing of a central longitudinal axis of the distal nasal-tool perforating tip by at least 15 degrees with respect to a central longitudinal axis of the nasal-guide channel without causing plastic deformation of the distal flexible portion.

For some applications, the distal flexible portion is sufficiently flexible to allow flexing of the central longitudinal axis of the distal nasal-tool perforating tip by at least 40 degrees with respect to the central longitudinal axis of the nasal-guide channel without causing plastic deformation of the distal flexible portion.

For some applications, the distal opening of the nasal-guide channel faces at least partially in a lateral direction that faces toward the lacrimal guide component.

For some applications, the nasal-guide channel is shaped so as to define a distal surface that faces partially in a proximal direction and partially in the lateral direction toward the lacrimal guide component so as to direct the distal nasal-tool perforating tip out of the distal opening of the nasal-guide channel, thereby flexing the distal flexible portion, when the nasal perforating tool is advanced distally through the nasal-guide channel.

For some applications, the distal flexible portion includes a coil.

For some applications:

the nasal-guide channel is configured to be both nasal-perforating-tool-accepting and guidewire-accepting,

the nasal perforating tool is shaped so as to define a nasal-perforating-tool guidewire-accepting channel,

the method further includes, after forming the bypass, placing a guidewire such that the guidewire passes through the lacrimal passageway, the bypass, and the entire nasal-guide channel, and out of a proximal end of the nasal-guide channel, and

advancing the nasal perforating tool within the nasal-guide channel until the distal nasal-tool perforating tip exits the distal opening of the distal nasal-tool perforating tip includes threading the guidewire into the nasal-perforating-tool guidewire-accepting channel and advancing the nasal perforating tool over the guidewire.

There is additionally provided, in accordance with an application of the present invention, a method of performing dacryocystorhinostomy (DCR), the method including:

inserting a nasal guide component of a dacryocystorhinostomy (DCR) guide into a nasal cavity of a patient's body, wherein the nasal guide component, including a laterally facing distal opening thereof, is shaped so as to define a nasal-guide channel;

forming a bypass between a lacrimal sac and the nasal cavity; and

thereafter, placing a guidewire such that the guidewire passes through a lacrimal passageway, the bypass, and at least a portion of the nasal-guide channel, the lacrimal passageway including punctum, a lacrimal canal, and a common canaliculus.

For some applications, placing the guidewire includes placing the guidewire such that the guidewire passes through the lacrimal passageway, the bypass, the entire nasal-guide channel, and out of a proximal end of the nasal-guide channel.

For some applications, placing the guidewire such that the guidewire passes through the lacrimal passageway includes:

advancing a lacrimal guidewire shaft through a lacrimal-guide channel of a lacrimal guide component of the DCR guide, the lacrimal passageway, and the bypass, until a distal tip of the lacrimal guidewire shaft is in contact with the laterally facing distal opening of nasal guide component, wherein the DCR guide constrains the laterally facing distal opening of nasal guide component to fall in a path of advancement of the distal tip; and

advancing the guidewire through a lacrimal guidewire-accepting channel of the lacrimal guidewire shaft while (a) the lacrimal guidewire shaft is disposed passing through the lacrimal passageway and the bypass, and (b) the distal tip of the lacrimal guidewire shaft is in contact with the laterally facing distal opening of nasal guide component.

For some applications, a distal opening of the nasal-guide channel faces at least partially in a lateral direction that faces toward the lacrimal guide component.

For some applications, inserting the nasal guide component and the lacrimal guide component includes using DCR guide to set a desired angle between respective central longitudinal axes of the nasal guide component and the lacrimal guidewire shaft.

For some applications, setting the desired angle includes using an arcuate portion of the DCR guide that allows relative movement between the nasal guide component and the lacrimal guide component to set the desired angle.

For some applications, placing the guidewire includes placing the guidewire such that a first end of the guidewire extends out of the patient's body through the lacrimal punctum and a second end of the guidewire, opposite the first end, extends out of the patient's body through the nasal cavity.

For some applications, the method further includes, after placing the guidewire:

removing the lacrimal guidewire shaft from the patient's body;

advancing a dilator along and over the guidewire and through the lacrimal passageway and into the bypass; and

dilating the bypass using the dilator.

For some applications, the dilator includes an inflatable element, and dilating the bypass includes inflating the inflatable element in the bypass.

For some applications, the method further includes, after dilating the bypass:

advancing a tubular support element along and over the guidewire and through the lacrimal passageway and into the bypass; and

removing the guidewire from the patient's body while leaving the tubular support element in place in the bypass.

There is yet additionally provided, in accordance with an application of the present invention, apparatus for performing dacryocystorhinostomy (DCR), the apparatus including a dacryocystorhinostomy (DCR) tool, which includes:

(a) a perforating shaft having a distal perforating-shaft perforating tip configured to form a bypass between a lacrimal sac and a nasal cavity through a lateral side of the lacrimal sac, a lacrimal bone, and nasal mucosa;

(b) a DCR guide, which includes:
(i) a nasal guide component, which is configured to be inserted into the nasal cavity and has a laterally facing distal opening, wherein the nasal guide component, including the laterally facing distal opening, is shaped so as to define a nasal-guide channel, which is shaped so as to define proximal and distal openings and configured to be nasal-perforating-tool-accepting; and
   (ii) a lacrimal guide component, which is shaped so as to define a lacrimal-guide channel that is configured to orient the DCR guide with respect to the distal perforating-shaft perforating tip during advancing of the distal perforating-shaft perforating tip through a lacrimal passageway and into the lacrimal sac, until contact of the distal perforating-shaft perforating tip with the internal wall of the nasal-shaft-accepting of the nasal guide component blocks further advancing of the distal perforating-shaft perforating tip, the lacrimal passageway including a lacrimal punctum, a lacrimal canal, and a common canaliculus,
      wherein the DCR guide is configured to constrain the laterally facing distal opening of the nasal guide component to fall in a path of advancement of the distal perforating-shaft perforating tip; and

(c) a nasal perforating tool, which is sized to pass through the nasal-guide channel, including the proximal and the distal openings thereof, and which includes:
(i) a distal nasal-tool perforating tip configured to widen the bypass between the lacrimal sac and the nasal cavity;
   (ii) a proximal elongate shaft; and
   (iii) a distal flexible portion disposed longitudinally between the proximal elongate shaft and the distal nasal-tool perforating tip.

For some applications, wherein the distal nasal-tool perforating tip is shaped as a drill bit.

For some applications, wherein the distal nasal-tool perforating tip is shaped as a punch.

For some applications, wherein the distal flexible portion includes a coil.

For some applications, wherein the DCR guide is configured to set a desired angle between respective central longitudinal axes of the nasal guide component and the perforating shaft.

For some applications, wherein the DCR guide is shaped so as to define an arcuate portion that is configured to allow relative movement between the nasal guide component and the lacrimal guide component to set the desired angle.

For some applications, wherein the distal perforating-shaft perforating tip is shaped as a drill bit.

For some applications, wherein the distal perforating-shaft perforating tip is shaped as a punch.

For some applications, wherein the nasal-guide channel is configured to be both nasal-perforating-tool-accepting and guidewire-accepting.

For some applications, wherein a distal opening of the nasal-guide channel faces at least partially in a lateral direction that faces toward the lacrimal guide component.

For some applications,

wherein the nasal guide component includes (a) an outer guide element that is shaped so as to define a nasal-shaft-accepting channel therethrough, and (b) a nasal shaft that is slidable through the nasal-shaft-accepting channel, and wherein the nasal shaft is shaped so as to define the laterally facing distal opening and the nasal-guide channel, and

wherein when the outer guide element and the nasal shaft are coupled together, a distal opening of the nasal-guide channel is constrained by the outer guide element and the nasal shaft to face at least partially in a lateral direction that faces toward the lacrimal guide component.

For some applications, wherein the nasal guide component includes a locking mechanism, which is configured to lock the nasal shaft rotationally with respect to the nasal-shaft-accepting channel, thereby maintaining the distal opening of the nasal-guide channel facing at least partially in the lateral direction.

For some applications, wherein the perforating shaft, including the distal perforating-shaft perforating tip, is shaped so as to define a lacrimal guidewire-accepting channel.

For some applications,

wherein the DCR tool further includes a lacrimal guidewire shaft having a distal tip, and wherein the lacrimal guidewire shaft, including the distal tip thereof, is shaped so as to define a lacrimal guidewire-accepting channel,

wherein the lacrimal-guide channel of the lacrimal guide component is configured to orient the DCR guide with respect to the distal tip of the lacrimal guidewire shaft during advancing of the distal tip through the lacrimal-guide channel and the lacrimal passageway and into the lacrimal sac, and

wherein the DCR guide is configured to constrain the laterally facing distal opening of the nasal guide component to fall in a path of advancement of the distal tip of the lacrimal guidewire shaft.

For some applications, wherein the DCR tool further includes a dilator, which is configured to be advanced through the lacrimal passageway and into the bypass, and to dilate the bypass.

For some applications, wherein the dilator includes an inflatable element, which is configured to dilate the bypass by being inflated in the bypass.

For some applications, the apparatus further includes a tubular support element, which is configured to be advanced through the lacrimal passageway and into the bypass, and to maintain patency of the bypass.

For some applications, wherein the nasal guide component includes (a) an outer guide element that is shaped so as to define a nasal-shaft-accepting channel therethrough, and (b) a nasal shaft that is slidable through the nasal-shaft-accepting channel, and wherein the nasal shaft is shaped so as to define the laterally facing distal opening.

For some applications, wherein the distal flexible portion is sufficiently flexible to allow flexing of a central longitudinal axis of the distal nasal-tool perforating tip by at least 15 degrees with respect to a central longitudinal axis of the nasal-guide channel when the distal nasal-tool perforating tip is advanced out of the distal opening of the nasal-guide channel while the nasal guide component is within the nasal cavity.

For some applications, wherein the distal flexible portion is sufficiently flexible to allow flexing of the central longitudinal axis of the distal nasal-tool perforating tip by at least 40 degrees with respect to the central longitudinal axis of the nasal-guide channel when the distal nasal-tool perforating tip is advanced out of the distal opening of the nasal-guide channel while the nasal guide component is within the nasal cavity.

For some applications, wherein the distal flexible portion is sufficiently flexible to allow flexing of a central longitudinal axis of the distal nasal-tool perforating tip by at least 15 degrees with respect to a central longitudinal axis of the nasal-guide channel without causing plastic deformation of the distal flexible portion.

For some applications, wherein the distal flexible portion is sufficiently flexible to allow flexing of the central longitudinal axis of the distal nasal-tool perforating tip by at least 40 degrees with respect to the central longitudinal axis of the nasal-guide channel without causing plastic deformation of the distal flexible portion.

For some applications, wherein the distal opening of the nasal-guide channel faces at least partially in a lateral direction that faces toward the lacrimal guide component.

For some applications, wherein the nasal-guide channel is shaped so as to define a distal surface that faces partially in a proximal direction and partially in the lateral direction toward the lacrimal guide component so as to direct the distal nasal-tool perforating tip out of the distal opening of the nasal-guide channel, thereby flexing the distal flexible portion, when the nasal perforating tool is advanced distally through the nasal-guide channel.

For some applications, wherein the nasal perforating tool is shaped so as to define a nasal-perforating-tool guidewire-accepting channel.

For some applications, wherein the nasal perforating tool is shaped such that the nasal-perforating-tool guidewire-accepting channel has a distal opening at a distal end of the distal nasal-tool perforating tip.

For some applications, wherein the dacryocystorhinostomy (DCR) tool device is for use with a guidewire, and wherein the distal flexible portion is sufficiently flexible to allow flexing of a central longitudinal axis of the distal nasal-tool perforating tip by at least 15 degrees with respect to a central longitudinal axis of the nasal-guide channel when the distal nasal-tool perforating tip is advanced over the guidewire and out of the distal opening of the nasal-guide channel while the nasal guide component is within the nasal cavity and the guidewire is within the nasal-perforating-tool guidewire-accepting channel.

For some applications, wherein the distal opening of the nasal-guide channel faces at least partially in a lateral direction that faces toward the lacrimal guide component.

For some applications, wherein the nasal-guide channel is shaped so as to define a distal surface that faces partially in a proximal direction and partially in the lateral direction toward the lacrimal guide component so as to direct the distal nasal-tool perforating tip out of the distal opening of the nasal-guide channel, thereby flexing the distal flexible portion, when the nasal perforating tool is advanced distally through the nasal-guide channel.

There is therefore provided, in accordance with an application of the present invention apparatus for performing dacryocystorhinostomy (DCR), the apparatus comprising a dacryocystorhinostomy (DCR) tool, which includes a perforating shaft having a distal perforating-shaft perforating tip configured to form a bypass between a lacrimal sac and a nasal cavity through a lateral side of the lacrimal sac, a lacrimal bone, and nasal mucosa; and a DCR guide, which includes a nasal guide component, which is configured to be inserted into the nasal cavity and has a nasal-guide channel having a proximal opening and an at least partially laterally facing distal opening; and

a lacrimal guide component, which is shaped so as to define a lacrimal-guide channel that is configured to orient the distal perforating-shaft perforating tip with respect to the DCR guide during advancing of the distal perforating-shaft perforating tip through a lacrimal passageway and into the lacrimal sac, until the distal perforating-shaft perforating tip at least crosses the laterally facing distal opening of the nasal guide component, wherein the DCR guide is configured to constrain the laterally facing distal opening of the nasal guide component to fall in a path of advancement of the distal perforating-shaft perforating tip.

For some applications, the DCR guide is configured to set a desired angle between respective central longitudinal axes of the nasal guide component and the laterally facing distal opening. In some applications the DCR guide is shaped so as to define an arcuate portion that is configured to allow relative movement between the nasal guide component and the lacrimal guide component to set the desired angle. In some applications, the distal perforating-shaft perforating tip is shaped as a drill bit. In some applications, the nasal-guide channel is configured to be guidewire-accepting.

For some applications, the distal opening of the nasal-guide channel faces at least partially in toward the lacrimal guide component. In some applications, the nasal guide component comprises (a) an outer guide element that is shaped so as to define a nasal-shaft-accepting channel therethrough, and (b) a nasal shaft that is slidable through the nasal-shaft-accepting channel, and wherein the nasal shaft is shaped so as to define a distal guide tip and the nasal-guide channel, and when the outer guide element and the nasal shaft are coupled together, a distal opening of the nasal-guide channel is constrained by the outer guide element and the nasal shaft to face at least partially in a lateral direction that faces toward the lacrimal guide component.

For some applications, the nasal guide component comprises a locking mechanism, which is configured to lock the nasal shaft with respect to the nasal-shaft-accepting channel, thereby maintaining the distal opening of the nasal-guide channel facing at least partially in the lateral direction. In some applications, wherein the perforating shaft, including the distal perforating-shaft perforating tip, is shaped so as to define a lacrimal guidewire-accepting channel. In some applications, the DCR tool further comprises a dilator, which is configured to be advanced through the nasal-shaft-accepting channel and laterally facing distal opening and into the bypass, and to dilate the bypass.

For some applications, the wherein the DCR guide is shaped so as to define an arcuate portion that is configured to allow relative movement between the nasal guide component and the lacrimal guide component to set a desired angle between respective central longitudinal axes of the nasal guide component and the perforating shaft in the lacrimal guide component, while maintaining a fixed point of intersection of the axes at the laterally facing distal opening. In some applications, the nasal guide component is shaped so as to define a nasal-guide channel, which is shaped so as to define proximal and distal openings and configured to be nasal-perforating-tool-accepting, and the DCR tool further comprises a nasal perforating tool, which is sized to pass through the nasal-guide channel, including the proximal and the distal openings thereof, and which includes a distal nasal-tool perforating tip configured to widen the bypass between the lacrimal sac and the nasal cavity, a proximal elongate shaft, and a distal flexible portion disposed longitudinally between the proximal elongate shaft and the distal nasal-tool perforating tip.

For some applications the distal nasal-tool perforating tip is shaped as a drill bit. In some applications, the nasal perforating tool is shaped so as to define a nasal-perforating-tool guidewire-accepting channel. In some applications, the nasal perforating tool is shaped such that the nasal-perforating-tool guidewire-accepting channel has a distal opening at a distal end of the distal nasal-tool perforating tip. In some applications, the dacryocystorhinostomy (DCR) tool device is for use with a guidewire, and wherein the distal flexible portion is sufficiently flexible to allow flexing of a central longitudinal axis of the distal nasal-tool perforating tip with respect to a central longitudinal axis of the nasal-guide channel when the distal nasal-tool perforating tip is advanced over the guidewire and out of the distal opening of the nasal-guide channel while the nasal guide component is within the nasal cavity and the guidewire is within the nasal-perforating-tool guidewire-accepting channel.

For some applications, the lacrimal guide component comprises a locking mechanism, which is configured to lock the perforating-shaft with respect to the lacrimal-guide channel of the lacrimal guide component.

There is therefore provided, in accordance with an application of the present invention a method of performing dacryocystorhinostomy (DCR), the method including inserting a nasal guide component of a dacryocystorhinostomy (DCR) guide into a nasal cavity of a patient's body the guide having a laterally facing distal opening, advancing a perforating shaft through a lacrimal-guide channel of a lacrimal guide component of the DCR guide and a lacrimal passageway and into a lacrimal sac, the lacrimal passageway including a lacrimal punctum, a lacrimal canal, and a common canaliculus, and forming a bypass between the lacrimal sac and the nasal cavity by advancing a distal perforating-shaft perforating tip through a lateral side of the lacrimal sac, a lacrimal bone, and nasal mucosa, until the distal perforating-shaft perforating tip at least enters the laterally facing distal opening, wherein the DCR guide constrains the laterally facing distal opening of the nasal guide component to fall in a path of advancement of the distal perforating-shaft perforating tip.

For some applications, the method also includes inserting the nasal guide component and the lacrimal guide component comprises using the DCR guide to set a desired angle between respective central longitudinal axes of the nasal guide component and the perforating shaft. In some applications, the method also includes setting the desired angle comprises using an arcuate portion of the DCR guide that allows relative movement between central longitudinal axes of the nasal guide component and the lacrimal guide component, while maintaining a fixed point of intersection of the axes at the laterally facing distal opening.

For some applications, the nasal guide component, including the distal guide tip, is shaped so as to define a nasal-guide channel, and wherein the method further comprises, after forming the bypass, placing a guidewire such that the guidewire passes through the lacrimal passageway, the bypass, and at least a portion of the nasal-guide channel.

The present invention will be more fully understood from the following detailed description of embodiments thereof, taken together with the drawings, in which:

### BRIEF DESCRIPTION OF THE DRAWINGS

FIG. 1 is a schematic illustration of a dacryocystorhinostomy (DCR) tool, in accordance with an application of the present invention;

FIGS. 2A-C are schematic illustrations of components of respective DCR tools, in accordance with respective applications of the present invention;

FIG. 3 is a flowchart illustrating a method of performing dacryocystorhinostomy (DCR), in accordance with an application of the present invention;

FIGS. 4A-I are schematic illustrations of the performance of the method of FIG. 3, in accordance with an application of the present invention;

FIGS. 5A-D are schematic illustrations of a locking mechanism of a nasal guide component of the DCR tool of FIG. 1, in accordance with an application of the present invention;

FIGS. 6A and 6B are schematic illustrations of another DCR tool, in accordance with an application of the present invention;

FIG. 7 is a schematic illustration of components of the DCR tool of FIGS. 6A and 6B, in accordance with an application of the present invention;

FIGS. 8A-G are schematic illustrations of a method for performing DCR using the DCR tool of FIGS. 6A, 6B, and 7, in accordance with an application of the present invention; and

FIG. 8H is a flowchart illustrating a method of performing dacryocystorhinostomy (DCR), in accordance with an application of the present invention.

### DETAILED DESCRIPTION OF APPLICATIONS

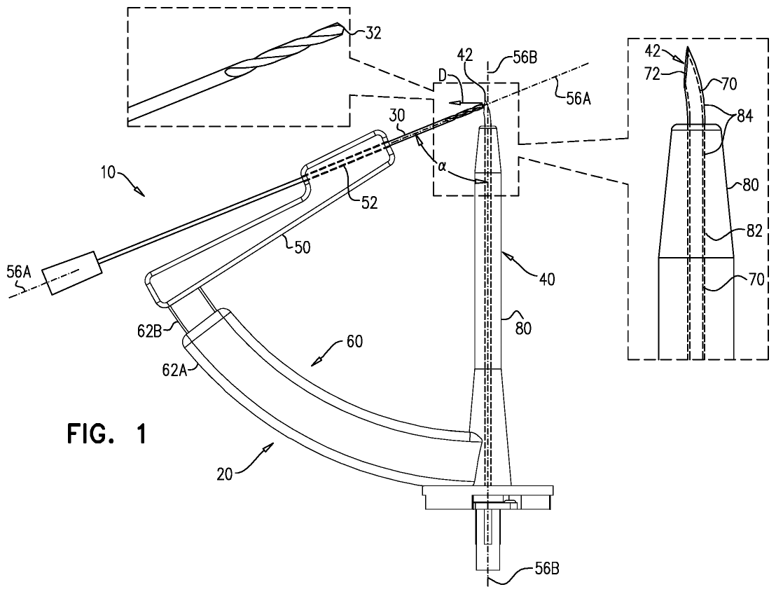

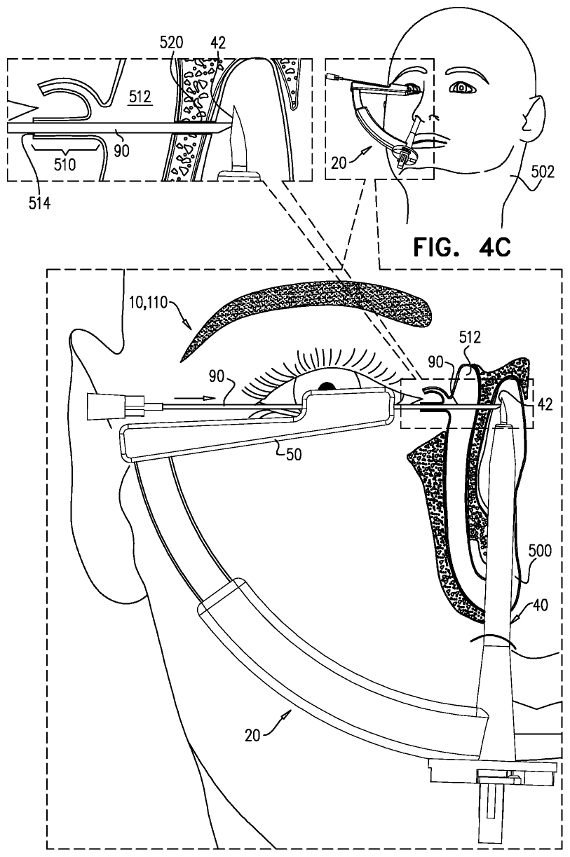

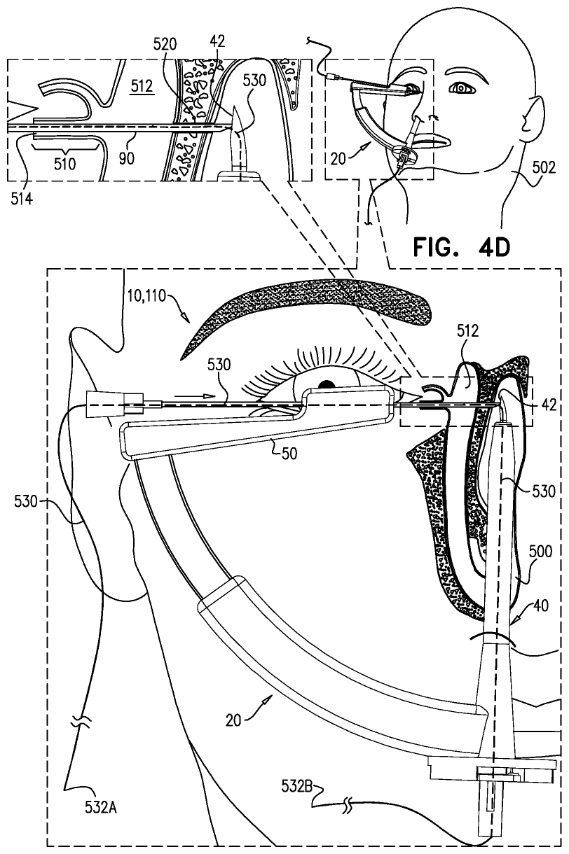

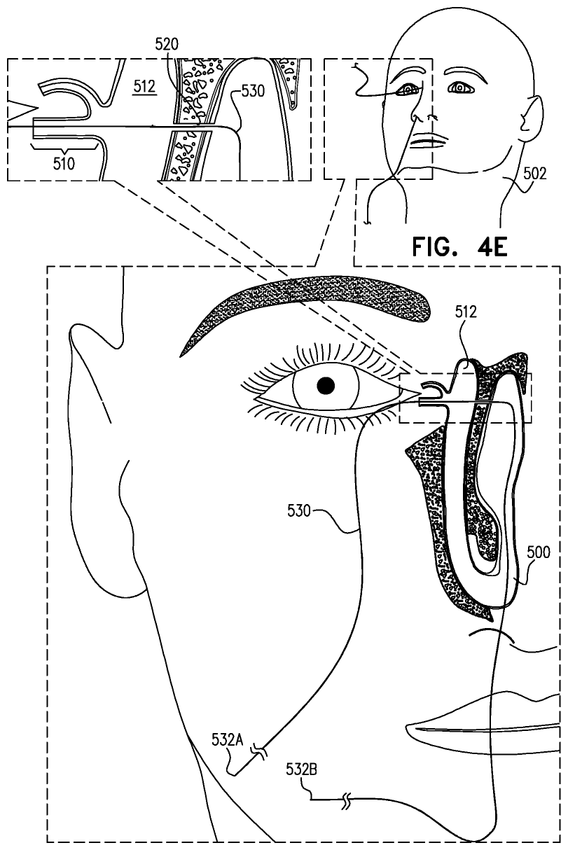

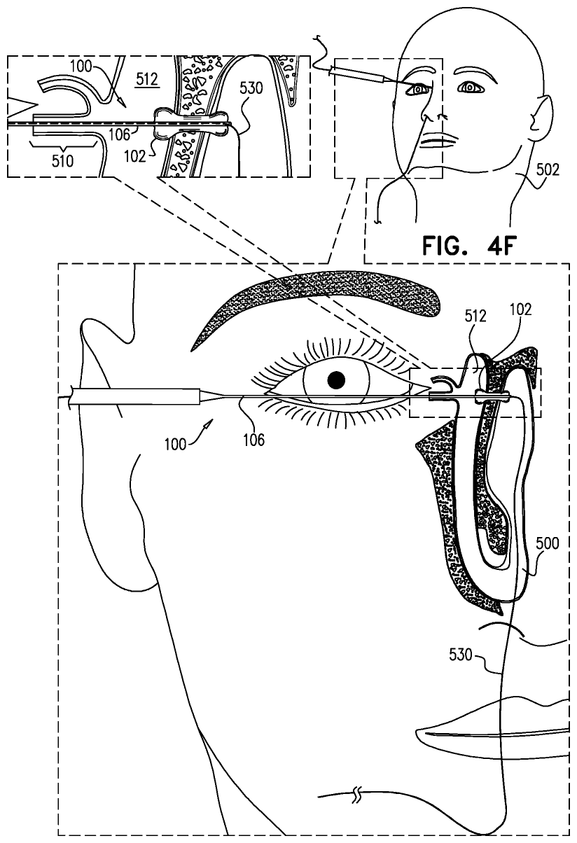

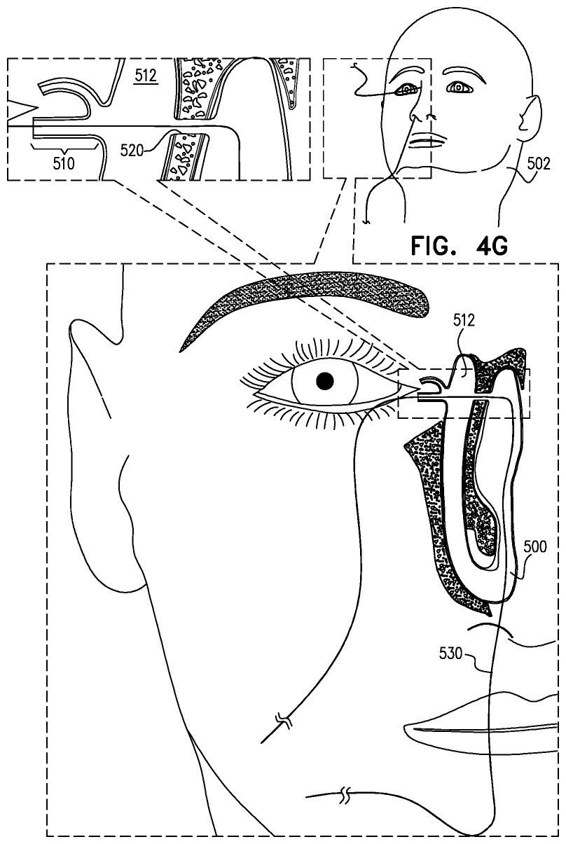

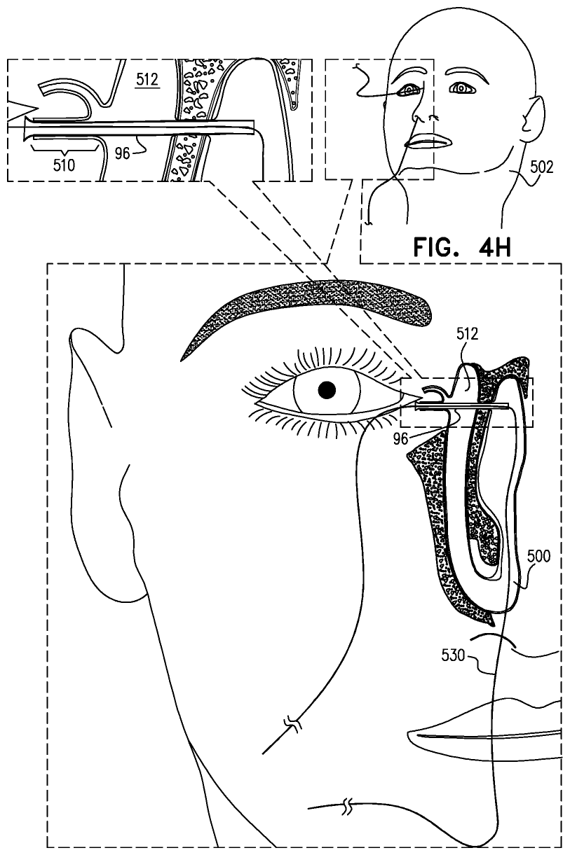

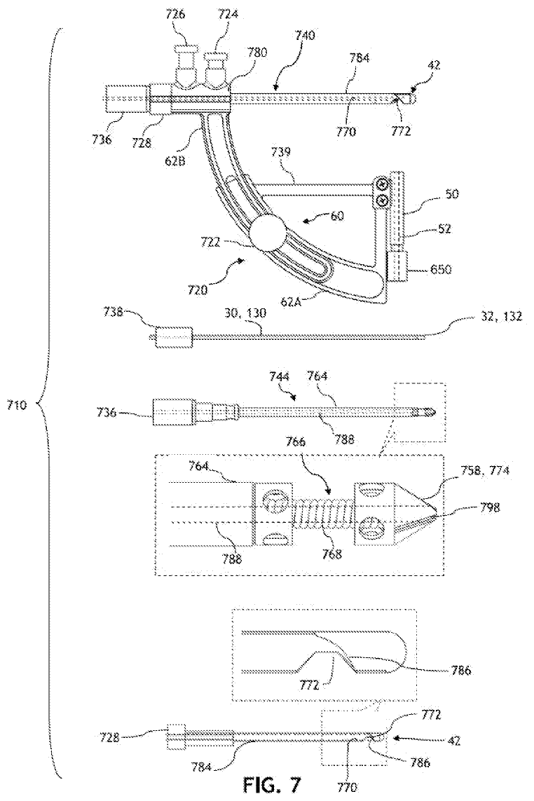

FIG. 1 is a schematic illustration of a dacryocystorhinostomy (DCR) tool 10, in accordance with an application of the present invention. DCR tool 10 is used to perform dacryocystorhinostomy (DCR), i.e., the formation of a bypass between the lacrimal system and the nasal cavity. DCR tool 10 comprises a dacryocystorhinostomy (DCR) guide 20 and, for some applications, a perforating shaft 30 having a proximal handle 738 (FIG. 7) and a distal perforating-shaft perforating tip 32 configured to form a bypass between a lacrimal sac and a nasal cavity through a lateral side of the lacrimal sac, a lacrimal bone, and nasal mucosa, such as described hereinbelow with reference to FIGS. 4A-B.

DCR guide 20 comprises:
a nasal guide component 40, which is configured to be inserted into the nasal cavity and has a laterally facing distal opening 42; and
a lacrimal guide component 50, which is shaped so as to define a lacrimal-guide channel 52, which is configured to orient DCR guide 20 (via lacrimal guide component 50) with respect to guide distal perforating-shaft perforating tip 32 during advancing of distal perforating-shaft perforating tip 32 through a lacrimal passageway and into a lacrimal sac, such as described hereinbelow with reference to FIG. 4A-B, until contact of distal perforating-shaft perforating tip 32 with a portion of the nasal-guide channel wall located opposite to the laterally facing distal opening 42 of nasal guide component 40 blocks further advancing of distal perforating-shaft perforating tip 32, such as described hereinbelow with reference to FIG. 4B. In some applications, perforating shaft 30 is fixed and lockable in place within lacrimal guide component 50.

DCR guide 20 is configured to constrain laterally facing distal opening 42 of the nasal guide component of nasal guide component 40 to fall in a path of advancement of distal perforating-shaft perforating tip 32. The lacrimal passageway to a large extent sets the path of advancement of distal perforating-shaft perforating tip 32, which in turn sets an orientation and location of perforating shaft 30. Perforating shaft 30 in turn sets an orientation and location of lacrimal guide component 50, which sets an orientation and location of nasal guide component 40, including laterally facing distal opening 42 of the nasal guide component, in the nasal cavity (as described in more detail hereinbelow with reference to FIG. 4B). As a result, laterally facing distal opening 42 is automatically and non-electrically positioned in the path of advancement of distal perforating-shaft perforating tip 32, and thus comes in contact with distal perforating-shaft perforating tip 32 and blocks its advancement. Typically, DCR guide 20 does not comprise any circuitry or other electrical or electronic elements.

Reference is still made to FIG. 1. For some applications, DCR guide 20 (e.g., a support structure thereof) is configured to set a desired angle α (alpha) between respective central longitudinal axes 56A and 56B of nasal guide component 40 and perforating shaft 30. For some of these applications, DCR guide 20 (e.g., a support structure thereof) is shaped so as to define an arcuate portion 60 that is configured to allow relative movement between nasal guide component 40 and lacrimal guide component 50 to set the desired angle α (alpha). For some applications, DCR guide 20 comprises first and second arcuate support members 62A and 62B, which are coupled in fixed orientation to nasal guide component 40 and lacrimal guide component 50, respectively, and together define arcuate portion 60. First and second arcuate support members 62A and 62B are arranged in slidable attachment with respect to one other so as to set a total aggregate length of an arc defined by the support members and thus angle α (alpha). For example, one of first and second arcuate support members 62A and 62B may be partially disposed within the other of first and second arcuate support members 62A and 62B, as shown, or first and second arcuate support members 62A and 62B be disposed alongside one another in slidable attachment.

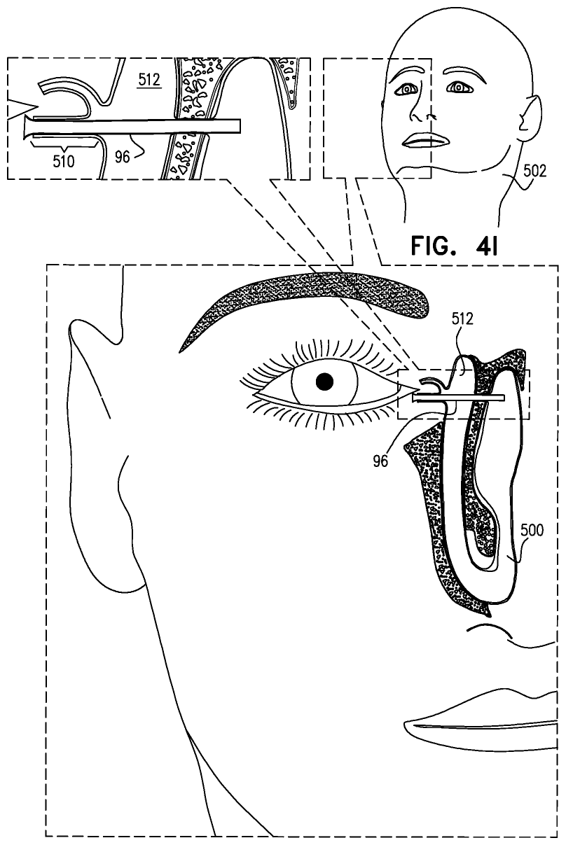

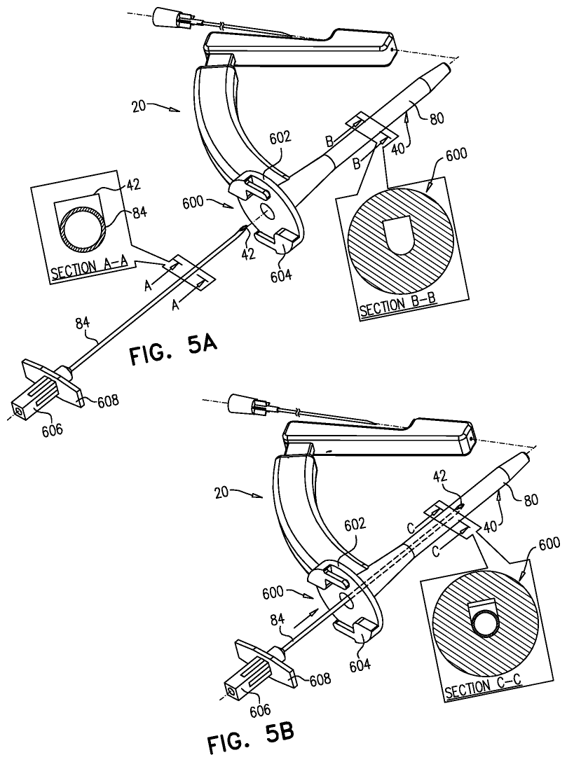

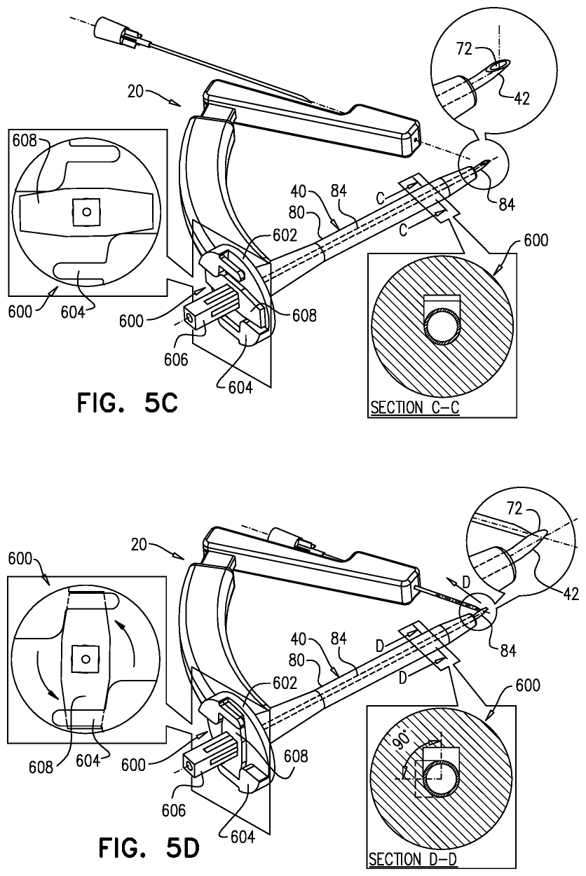

Reference is still made to FIG. 1. For some applications, nasal guide component 40, including laterally facing distal opening 42, is shaped so as to define a nasal-guide channel 70, which is configured to be guidewire-accepting, i.e., sized to accept insertion of guidewire 530, described hereinbelow with reference to FIGS. 4C-E. Typically, a laterally facing distal opening 42 of nasal-guide channel 70 faces at least partially in a lateral direction D that faces toward lacrimal guide component 50 (typically, this orientation is set (i.e., constrained) by DCR guide 20, such as described hereinbelow with reference to FIGS. 5A-D). (As used in the present application, including in the claims, the phrase “faces toward” does not require the lateral direction D to be directed entirely toward lacrimal guide component 50.)

Reference is still made to FIG. 1. For some applications, nasal guide component 40 comprises (a) an outer guide element 80 that is shaped so as to define a nasal-shaft-accepting channel 82 therethrough, and (b) a nasal shaft 84 that is slidable through nasal-shaft-accepting channel 82, typically before nasal guide component 40 is inserted into the nasal cavity. Nasal shaft 84 is shaped so as to define laterally facing distal opening 42 and nasal-guide channel 70, if provided. For some applications, as shown, nasal shaft 84 comprises a needle, which, for example, may be a Tuohy needle, as is known in the epidural art. As mentioned above, laterally facing distal opening 42 of nasal-guide channel 70 typically faces at least partially in lateral direction D. For some applications, DCR guide 20 is configured to rotationally orient laterally facing distal opening 42 such that lateral direction D faces toward lacrimal guide component 50. For some applications, nasal guide component 40 further comprises a locking mechanism 600, such as described hereinbelow with reference to FIGS. 5A-D.

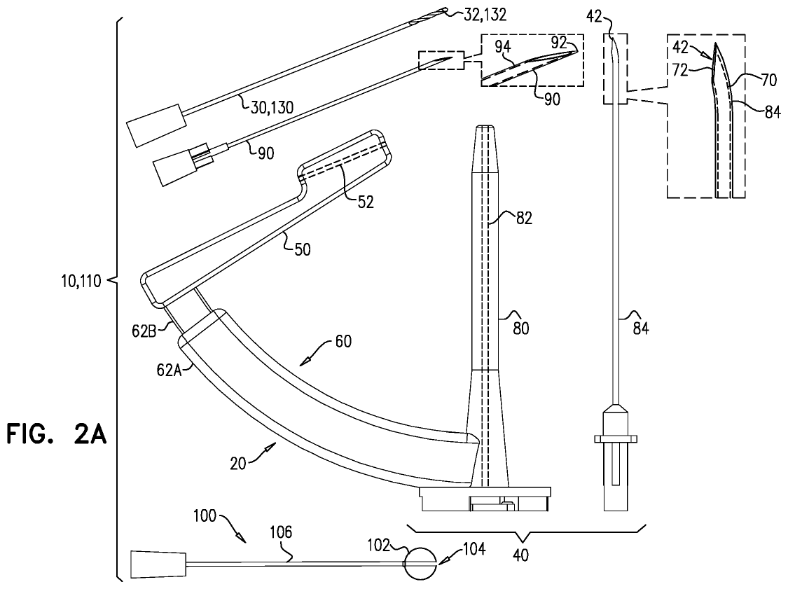

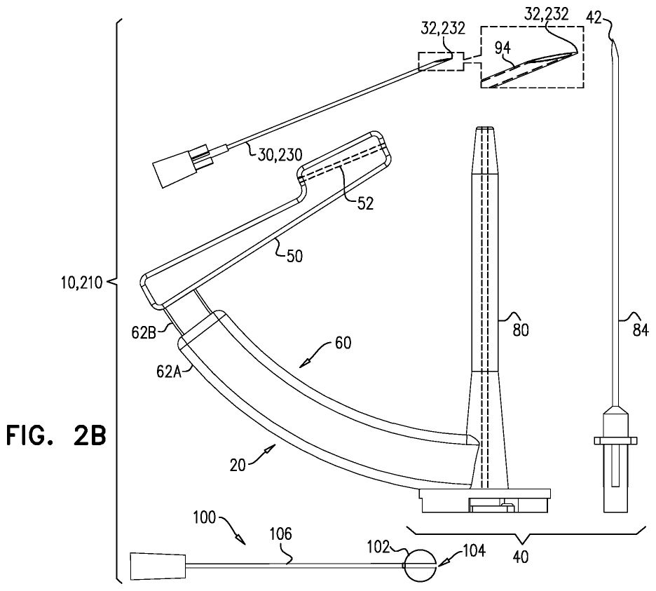

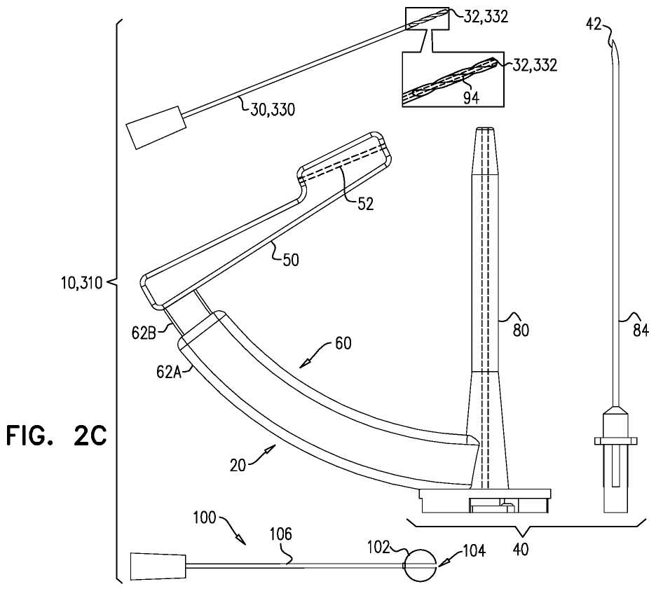

Reference is now made to FIGS. 2A-C, which are schematic illustrations of components of DCR tools 110, 210, and 310, respectively, in accordance with respective applications of the present invention. DCR tools 110, 210, and 310 are implementations of DCR tool 10, described hereinabove with reference to FIG. 1 and may implement any of the features thereof.

For some applications, DCR tool 10 further comprises a dilator 100, which is configured to be advanced through the lacrimal passageway and into the bypass, and to dilate the bypass. For some of these applications, dilator 100 comprises an inflatable element 102, such as a balloon, which is configured to dilate the bypass by being inflated in the bypass. Typically, inflatable element 102 is disposed at or near a distal end 104 of a shaft 106 of dilator 100. Dilator 100 is typically inflated about 12 mm medially from a lacrimal punctum 514, and typically has a length of 10-15 mm. For other applications, DCR tool 10 does not comprise dilator 100.

Reference is made to FIG. 2A. In this configuration, DCR tool 110 further comprises, in addition to perforating shaft 30, a lacrimal guidewire shaft 90 having a distal tip 92 (which is either sharp, as shown, or blunt). In this configuration, perforating shaft 30 is typically solid, i.e., does not define a channel therethrough. For some applications, perforating shaft 30 comprises a drilling perforating shaft 130, and distal perforating-shaft perforating tip 32 is shaped as a drill bit 132. For other applications, perforating shaft 30 comprises a punching perforating shaft, and distal perforating-shaft perforating tip 32 is shaped as a punch (configuration not shown, but similar to punching perforating shaft 230 described hereinbelow with reference to FIG. 2B, except that in the present configuration the punching perforating shaft does not necessarily define a channel therethrough). Further alternatively, perforating shaft 30 comprises an energy-application perforating shaft, which uses energy (e.g., RF, electrical, or laser) to form the bypass.

Reference is still made to FIG. 2A. Lacrimal guidewire shaft 90, including distal tip 92 thereof, is shaped so as to define a lacrimal guidewire-accepting channel 94. lacrimal-guide channel 52 of lacrimal guide component 50 is configured to orient DCR guide 20 (via lacrimal guide component 50) with respect to distal tip 92 of lacrimal guidewire shaft 90 during advancing of distal tip 92 through lacrimal-guide channel 52 and the lacrimal passageway and into the lacrimal sac. DCR guide 20 is configured to constrain laterally facing distal opening 42 of nasal guide component 40 to fall in a path of advancement of distal tip 92 of lacrimal guidewire shaft 90.

Reference is still made to FIG. 2A. For some applications, DCR tool 110 does not comprise perforating shaft 30. For these applications, the surgeon forms bypass using a perforating tool that is not a component of DCR tool 110, either using or not using DCR tool 110.

Reference is made to FIGS. 2B and 2C. In these configurations, perforating shaft 30, including distal perforating-shaft perforating tip 32, is shaped so as to define lacrimal guidewire-accepting channel 94. In the configuration shown in FIG. 2B, perforating shaft 30 of DCR tool 220 comprises a punching perforating shaft 230, and distal perforating-shaft perforating tip 32 is shaped as a punch 232. In the configuration shown in FIG. 2C, perforating shaft 30 of DCR tool 320 comprises a drilling perforating shaft 330, and distal perforating-shaft perforating tip 32 is shaped as a drill bit 332.

For some applications, a tubular support element 96 is provided, which is configured to be advanced through the lacrimal passageway and into the bypass, and to maintain patency of the bypass, such as described hereinbelow with reference to FIG. 4H.

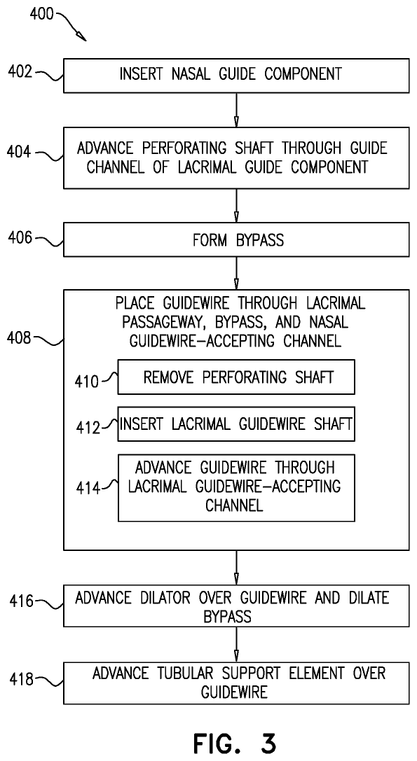

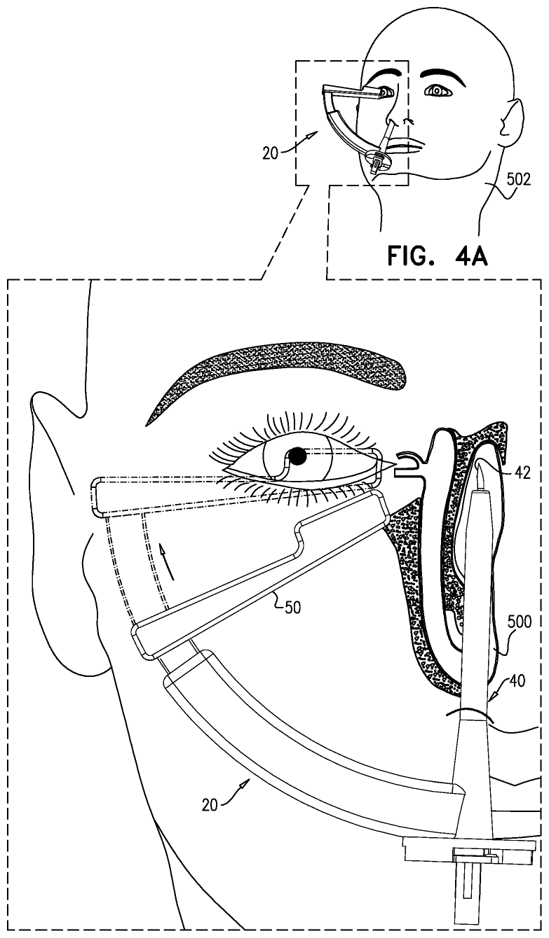

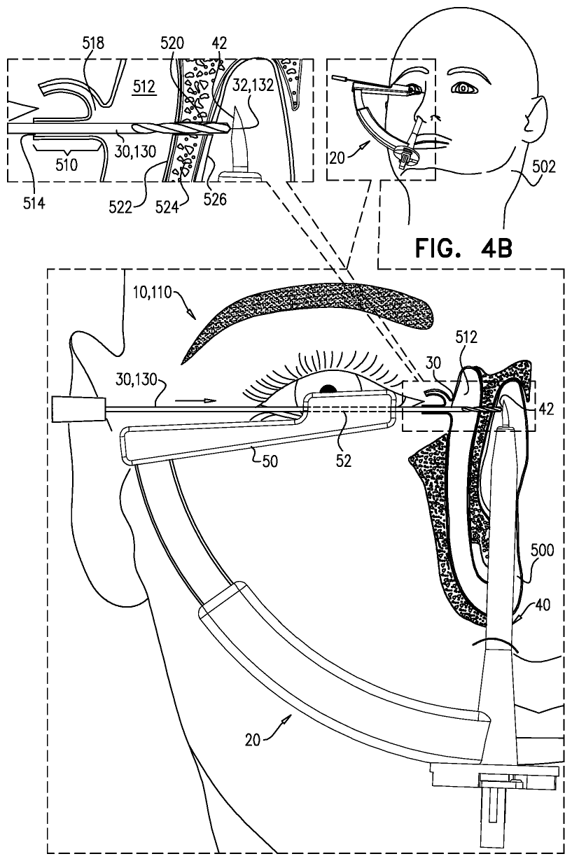

Reference is now made to FIG. 3, which is a flowchart illustrating a method 400 of performing dacryocystorhinostomy (DCR), in accordance with an application of the present invention. Reference is also made to FIGS. 4A-I, which are schematic illustrations of the performance of method 400, in accordance with an application of the present invention. Method 400 is illustrated using DCR tool 110, described hereinabove with reference to FIG. 2A. DCR tool 210 or DCR tool 310, described hereinabove with reference to FIGS. 2B and 2C, respectively, may alternatively be used, mutatis mutandis, such as described hereinbelow.

As shown in FIG. 4A, at a nasal guide insertion step 402, nasal guide component 40 of DCR guide 20 is inserted into a nasal cavity 500 of a patient's body 502. Nasal guide component 40 need not be inserted precisely by the surgeon, so long as it is inserted into the correct nostril, because it will be precisely oriented and positioned by DCR guide 20 at lacrimal advancement step 404, described hereinbelow with reference to FIG. 4B.

As shown in FIG. 4B, at a lacrimal advancement step 404, perforating shaft 30 is advanced through lacrimal-guide channel 52 of lacrimal guide component 50 of DCR guide 20 and a lacrimal passageway 510 and into a lacrimal sac 512. Lacrimal passageway 510 includes lacrimal punctum 514 (either inferior, as shown, or superior, not shown), a lacrimal canal 516 (either inferior, as shown, or superior, not shown), and a common canaliculus 518. Surgeons skilled in the DCR art generally are able to advance perforating shaft 30 through lacrimal passageway 510 without difficulty.

DCR guide 20 constrains laterally facing distal opening 42 of nasal guide component 40 to fall in a path of advancement of distal perforating-shaft perforating tip 32. As a result of this constraint, DCR guide 20 typically positions laterally facing distal opening 42 of nasal guide component 40 at an axilla of a middle turbinate of nasal cavity 500. Optionally, the surgeon may visually confirm the proper positioning of laterally facing distal opening 42, such as using a nasal endoscope.

For some applications, such as shown in the transition between FIG. 4A and FIG. 4B, for performing nasal guide insertion step 402 and lacrimal advancement step 404, DCR guide 20 is used to set a desired angle α (alpha) between respective central longitudinal axes 56A and 56B of nasal guide component 40 and perforating shaft 30, such as described hereinabove with reference to FIG. 1, based on the particular anatomy of the patient (e.g., the shape and size of the relevant parts of the anatomy). For some applications, the desired angle α (alpha) is set using arcuate portion 60 of DCR guide 20 that allows relative movement between nasal guide component 40 and lacrimal guide component 50 to set the desired angle, such as described hereinabove with reference to FIG. 1.

For some applications, perforating shaft 30 is advanced through at least a portion of lacrimal passageway 510 (and optionally into lacrimal sac 512) before perforating shaft 30 is placed within lacrimal-guide channel 52 of lacrimal guide component 50. For example, lacrimal-guide channel 52 may have a lateral elongate axial opening (e.g., a slit) that allows lateral insertion of perforating shaft 30 into lacrimal guide component 50 even after perforating shaft 30 has been placed within the at least a portion of lacrimal passageway 510.

Also as shown in FIG. 4B, at a perforation step 406, a bypass 520 is formed between lacrimal sac 512 and nasal cavity 500 by advancing distal perforating-shaft perforating tip 32 of perforating shaft 30 through a lateral side 522 of lacrimal sac 512, a lacrimal bone 524, and nasal mucosa 526, until contact of distal perforating-shaft perforating tip 32 with laterally facing distal opening 42 of nasal guide component 40 blocks further advancing of distal perforating-shaft perforating tip 32. This contact prevents over-advancement distal perforating-shaft perforating tip 32, which might otherwise perforate tissue across nasal cavity 500, which is generally no more than several millimeters beyond bypass 520. For some applications, such as shown in FIG. 4B, distal perforating-shaft perforating tip 32 is drilled through the lateral side of lacrimal sac 512, lacrimal bone 524, and nasal mucosa 526. Alternatively, distal perforating-shaft perforating tip 32 is punched through the lateral side of lacrimal sac 512, lacrimal bone 524, and nasal mucosa 526 (technique not shown in FIG. 4B).

Lacrimal advancement step 404 and perforation step 406 are optional; the surgeon may instead form bypass 520 using a perforating tool that is not a component of DCR tool 110 (e.g., either mechanically or using energy, e.g., laser energy), either using or not using DCR tool 110.

For some applications, as shown in FIGS. 4C-E, at a guidewire placement step 408, a guidewire 530 is placed such that guidewire 530 passes through lacrimal passageway 510, bypass 520, and at least a portion of nasal-guide channel 70 (labeled in FIGS. 1 and 2A). (In these applications, nasal guide component 40, including laterally facing distal opening 42, is shaped so as to define nasal-guide channel 70.) Typically, guidewire 530 is advanced in a direction from lacrimal punctum 514 toward nasal cavity 500. Typically, guidewire 530 is placed such that guidewire 530 passes through lacrimal passageway 510, bypass 520, the entire nasal-guide channel 70, and out of a proximal end of nasal-guide channel 70. Typically, laterally facing distal opening 42 of nasal-guide channel 70 faces at least partially in lateral direction D that faces toward lacrimal guide component 50, such as described hereinabove with reference to FIG. 1.

Optionally, guidewire 530 has a diameter of 0.15-0.5 mm, e.g., 0.2-0.25 mm.

For some applications, guidewire placement step 408 comprises:
at a perforating shaft removal step 410, removing perforating shaft 30 from the patient's body (after forming bypass 520, as shown in FIG. 4B);
as shown in FIG. 4C, at a lacrimal guidewire insertion step 412, inserting lacrimal guidewire shaft 90 through lacrimal-guide channel 52 of lacrimal guide component 50 and lacrimal passageway 510 and into lacrimal sac 512; DCR guide 20 constrains laterally facing distal opening 42 of nasal guide component 40 to fall in a path of advancement of distal tip 92 of lacrimal guidewire shaft 90 (as describe hereinabove with reference to FIG. 2A, lacrimal guidewire shaft 90, including distal tip 92 thereof, is shaped so as to define lacrimal guidewire-accepting channel 94);
as shown in FIG. 4D, at a guidewire advancement step 414, advancing guidewire 530 through lacrimal guidewire-accepting channel 94 while lacrimal guidewire shaft 90 is disposed passing through lacrimal passageway 510; and
withdrawing lacrimal guide component 50, lacrimal guidewire shaft 90, and nasal guide component 40 from guidewire 530 and out of the patient's body, leaving guidewire 530 in place, as shown in FIG. 4E, typically such that a first end 532A of guidewire 530 extends out of the patient's body through lacrimal punctum 514 and a second end 532B of guidewire 530, opposite first end 532A, extends out of the patient's body through nasal cavity 500.

Typically, as shown in FIG. 4D, at guidewire advancement step 414, guidewire 530 is advanced through lacrimal guidewire-accepting channel 94 while (a) lacrimal guidewire shaft 90 is disposed passing through lacrimal passageway 510 and bypass 520, and (b) distal tip 92 of lacrimal guidewire shaft 90 is in contact with laterally facing distal opening 42 of nasal guide component 40.

For some applications, as shown in FIG. 4F, at a dilation step 416, dilator 100 is advanced along and over guidewire 530 and through lacrimal passageway 510 and into bypass 520, and bypass 520 is dilated using dilator 100. As shown in FIG. 4G, dilator 100 is removed from the patient's body.

For some applications, such as shown in FIG. 4H, at a support step 418, tubular support element 96 is advanced along and over guidewire 530 and through lacrimal passageway 510 and into bypass 520, and guidewire 530 is removed from the patient's body while leaving the tubular support element in place in bypass 520, as shown in FIG. 4I. For example, tubular support element 96 may comprise a stent (e.g., comprising metal (e.g., Nitinol) and/or a polymer, e.g., silicone) or a polymer tube, e.g., comprising silicone. For some applications, tubular support element 96 is left in place long-term, while for other applications, tubular support element 96 is removed after patency of bypass 520 is achieved, such as after a few weeks. Although the proximal end of tubular support element 96 is shown as terminating outside lacrimal punctum 514, tubular support element 96 may alternatively be shorter, and terminate within lacrimal canal 516, common canaliculus 518, in lacrimal sac 512.

Reference is made to FIGS. 2B-C and FIG. 4B-E. For applications in which DCR tool 210, described hereinabove with reference to FIG. 2B, or DCR tool 310, described hereinabove with reference to FIG. 2C, is used to perform the DCR, guidewire placement step 408 does not comprise perforating shaft removal step 410; instead, perforating shaft 30 is left in lacrimal passageway 510 after forming bypass 520. At lacrimal guidewire insertion step 412, guidewire 530 is advanced through lacrimal guidewire-accepting channel 94 while perforating shaft 30 is disposed passing through lacrimal passageway 510, such that guidewire 530 passes through lacrimal passageway 510. Typically, guidewire 530 is advanced through lacrimal guidewire-accepting channel 94 while (a) perforating shaft 30 is disposed passing through lacrimal passageway 510 and bypass 520, and (b) distal perforating-shaft perforating tip 32 is in contact with laterally facing distal opening 42 of nasal guide component 40. Thereafter, lacrimal guide component 50, perforating shaft 30, and nasal guide component 40 are withdrawn from guidewire 530 and out of the patient's body, leaving guidewire 530 in place, as shown in FIG. 4E.

Reference is now made to FIGS. 5A-D, which are schematic illustrations of a locking mechanism 600 of nasal guide component 40, in accordance with an application of the present invention. In this configuration, as mentioned above with reference to FIG. 1, nasal guide component 40 comprises (a) outer guide element 80 that is shaped so as to define nasal-shaft-accepting channel 82 therethrough (labeled in FIG. 2A), and (b) nasal shaft 84 that is slidable through nasal-shaft-accepting channel 82, typically before nasal guide component 40 is inserted into the nasal cavity. Nasal shaft 84 is shaped so as to define laterally facing distal opening 42 and nasal-guide channel 70 (labeled in FIG. 2A). Providing nasal shaft 84 as a component separate from outer guide element 80 (and from the other elements of DCR guide 20) may enable reusability of DCR guide 20 with a plurality of disposable nasal shafts 84 (for example, because nasal shaft 84 may become slightly damaged, e.g., bent, during the surgical procedure).

Typically, as shown in FIG. 5D, when outer guide element 80 and nasal shaft 84 are coupled together, laterally facing distal opening 42 of nasal-guide channel 70 is constrained by outer guide element 80 and nasal shaft 84 to face at least partially in lateral direction D, described hereinabove with reference to FIG. 1.

Optionally, but not necessarily, nasal guide component 40 comprises locking mechanism 600, which is configured to lock nasal shaft 84 rotationally with respect to nasal-shaft-accepting channel 82, thereby maintaining the rotational orientation of laterally facing distal opening 42 of nasal-guide channel 70, i.e., facing at least partially in lateral direction D after rotationally orienting laterally facing distal opening 42, as shown in FIG. 5D. Locking mechanism 600 also typically locks nasal shaft 84 axially with respect to nasal-shaft-accepting channel 82, which fixes the axial position of laterally facing distal opening 42 with respect to nasal guide component 40 and ensures that laterally facing distal opening 42 falls in the path of advancement of distal tip 92 of lacrimal guidewire shaft 90, as described hereinabove with reference to FIG. 1.

FIG. 5A shows DCR guide 20 before insertion of nasal shaft 84 into nasal-shaft-accepting channel 82 (labeled in FIG. 2A) of outer guide element 80 of nasal guide component 40. FIG. 5B shows DCR guide 20 upon partial insertion of nasal shaft 84 into nasal-shaft-accepting channel 82. FIG. 5C shows DCR guide 20 after insertion of nasal shaft 84 into nasal-shaft-accepting channel 82, while locking mechanism 600 is in an unlocked state.

For some applications, as shown in FIG. 5D, rotation of nasal shaft 84 with respect to outer guide element 80 transitions locking mechanism 600 from the unlocked state to a locked state, in which laterally facing distal opening 42 of nasal-guide channel 70 is constrained and locked to face at least partially in lateral direction D. In addition, in the locked state, locking mechanism 600 locks nasal shaft 84 axially with respect to nasal-shaft-accepting channel 82.

For some applications, as shown in FIGS. 5A-D, nasal guide component 40 comprises a first proximal base 602 that is shaped so as to define a first coupling element 604 of locking mechanism 600, and nasal shaft 84 comprises a second proximal base 606 that is shaped so as to define a second coupling element 608 of locking mechanism 600. First and second coupling elements 604 and 608 are configured to be locked together, such as by rotation with respect to each other, as shown in FIG. 5D.

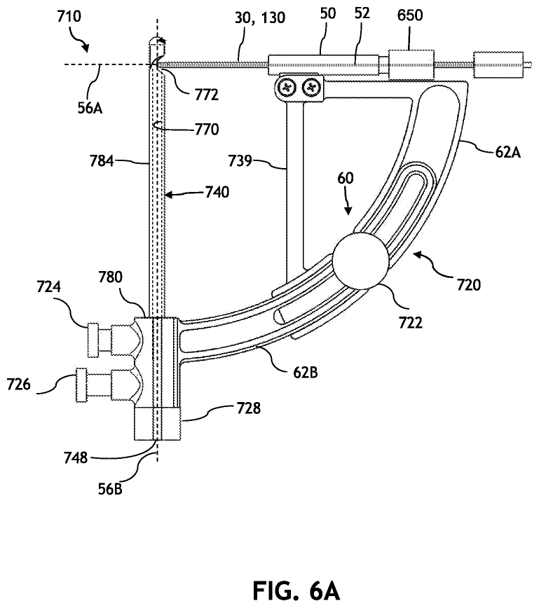

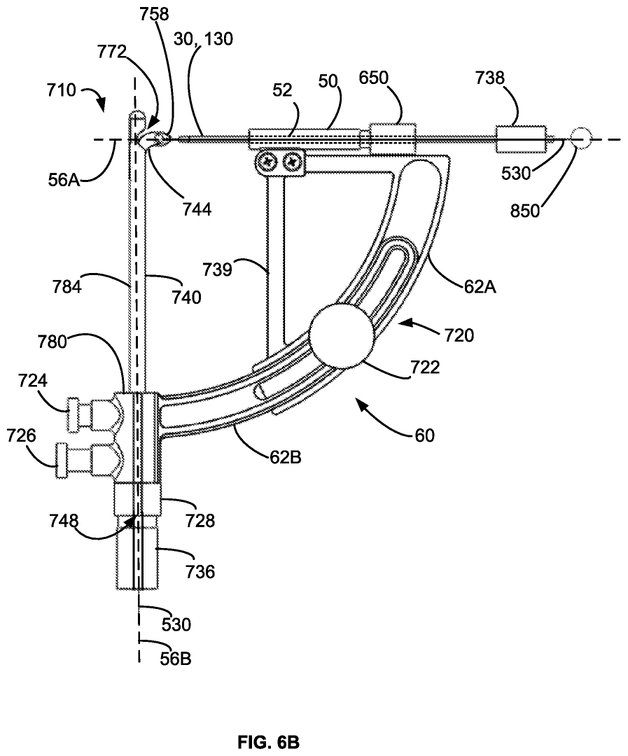

Reference is now made to FIGS. 6A and 6B, which are schematic illustrations of a dacryocystorhinostomy (DCR) tool 710, in accordance with an application of the present invention. DCR tool 710 is used to perform dacryocystorhinostomy (DCR), i.e., the formation of a bypass between the lacrimal system and the nasal cavity. Other than as described below, DCR tool 710 is similar to DCR tool 10 described hereinabove, and may implement any of the techniques described hereinabove for DCR tool 10 like reference numerals refer to like or similar parts.

DCR tool 710 comprises a dacryocystorhinostomy (DCR) guide 720 and, for some applications, perforating shaft 30, as shown in FIG. 6A. In some applications, perforating shaft 30 is slidable through a lacrimal-guide channel 52 inside a lacrimal guide component 50, which is configured to orient DCR guide 720 (via lacrimal guide component 50) with respect to guide distal perforating-shaft 30 perforating tip during advancing of distal perforating-shaft perforating tip through a lacrimal passageway and into a lacrimal sac, Other than as described below, DCR guide 720 comprises the same elements as DCR guide 20, described hereinabove.

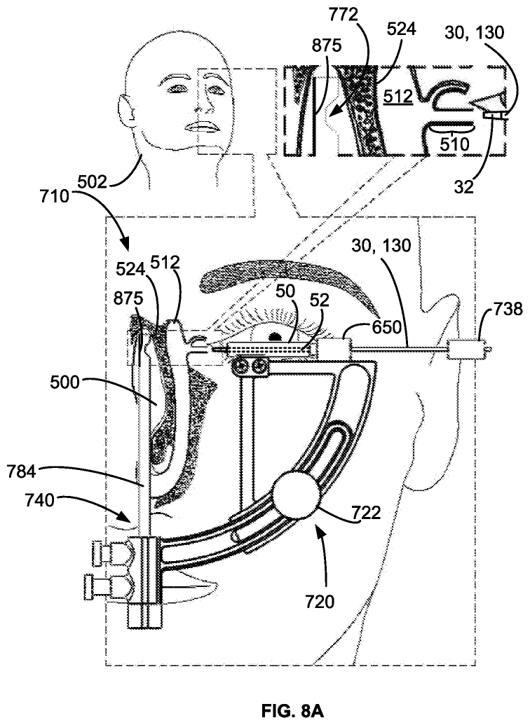

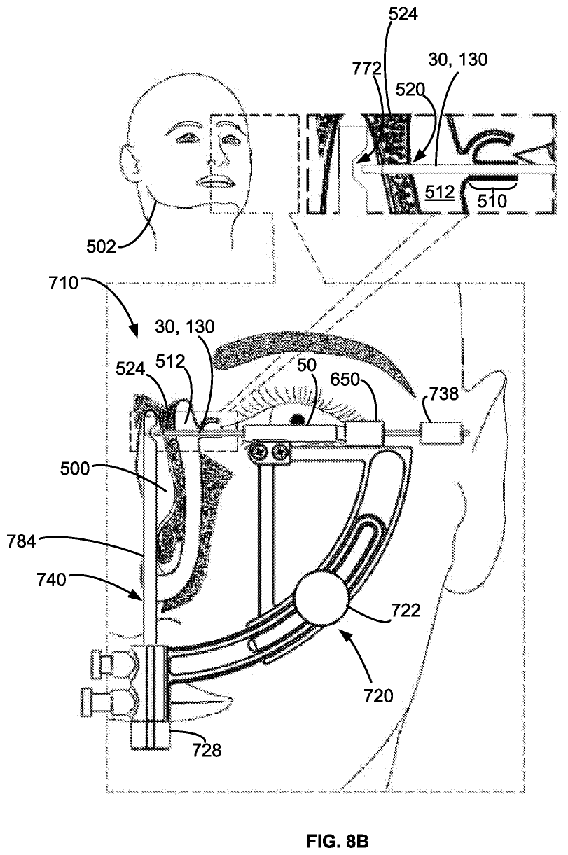

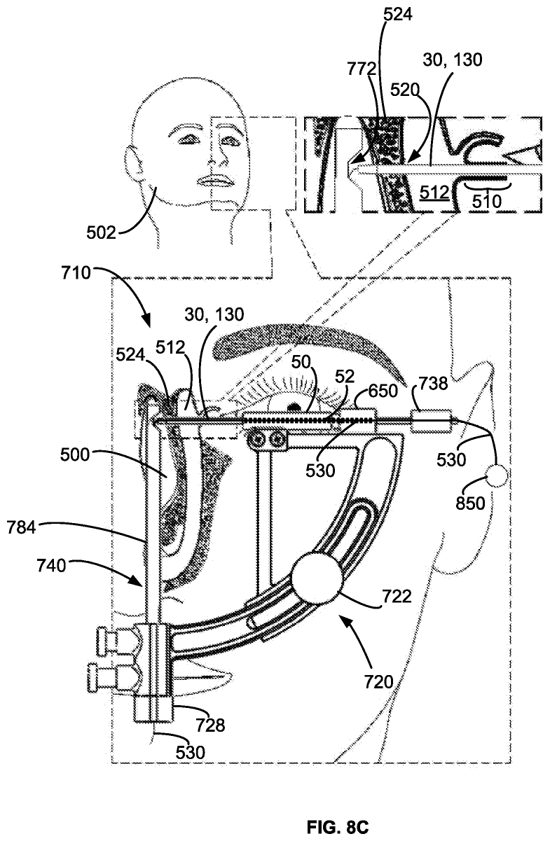

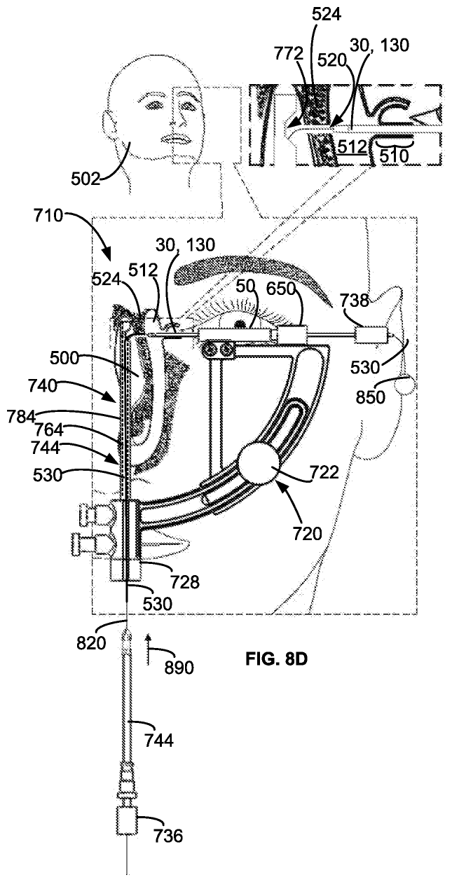

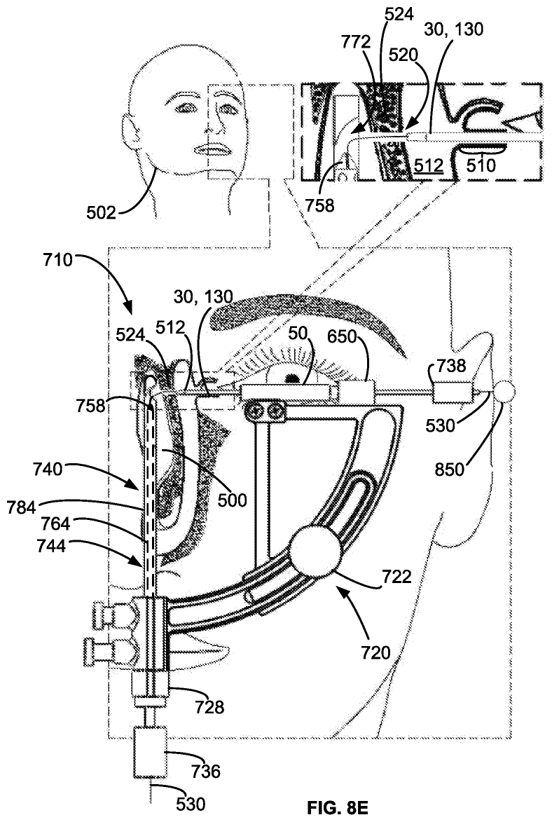

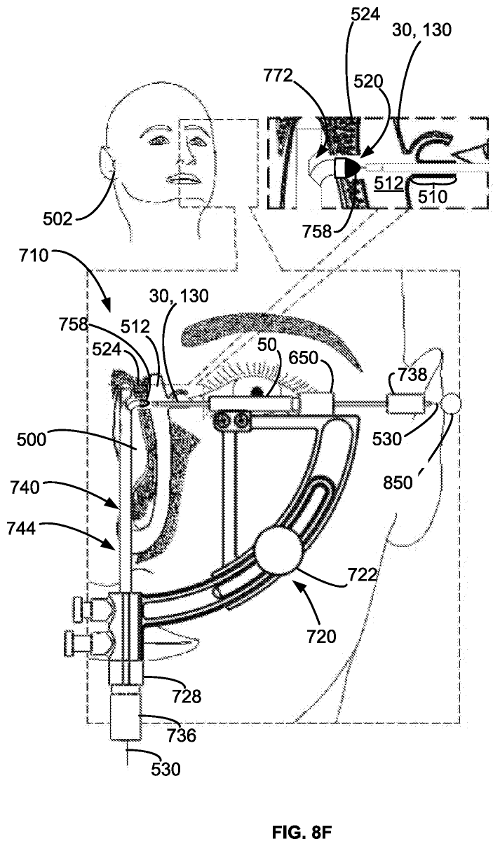

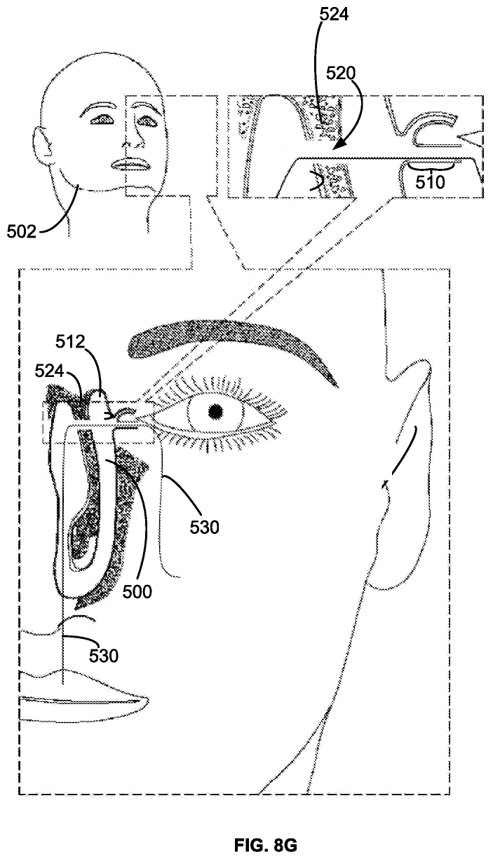

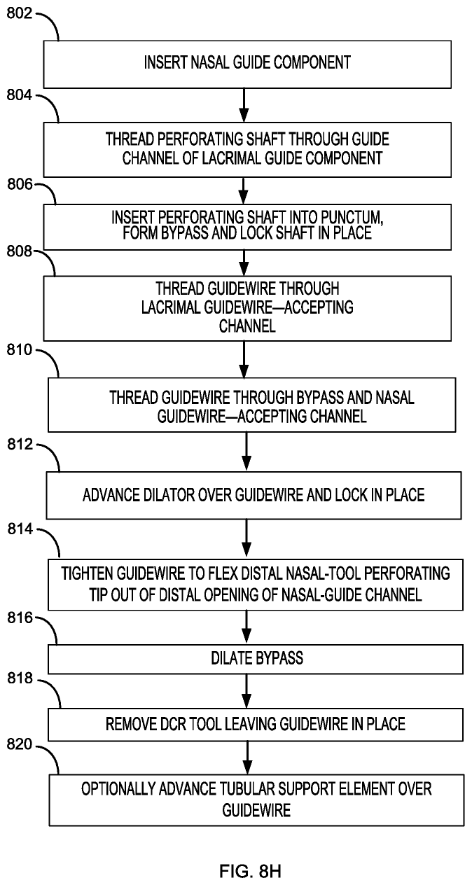

FIG. 6B shows DCR tool 710 after perforating shaft 30 has been slightly retracted, such as described hereinbelow with reference to FIG. 8F. As described in greater detail below, distal nasal-tool perforating tip 758 is not advanced or urged out of laterally facing distal opening 772. Distal nasal-tool perforating tip 758 is directed out of opening 772 by virtue of tension alone placed on guidewire 530 being pulled out of proximal handle 736 nasal perforating tool 744. Pulling on guidewire 530 not only places tension on it, but also shortens guidewire 530 length between proximal handle 736 and stopper 850 thus flexing distal nasal-tool perforating tip 758 out of distal laterally facing opening 772. Hence distal nasal-tool perforating tip 758 flexes passively and conforms to the shortening and therefore change of the angle of guidewire 530 in the section between distal laterally facing opening 772 of nasal-guide channel 770 and distal perforating-shaft perforating tip 32.

As shown in FIG. 6B, DCR guide 720 comprises a nasal guide component 740, which is configured to be inserted into the nasal cavity and has laterally facing distal opening 42. Nasal guide component 740 may implement any of the features of nasal guide component 40, described hereinabove, mutatis mutandis. Nasal guide component 740, including laterally facing distal opening 42, is shaped so as to define a nasal-guide channel 770, which is configured to be nasal-perforating-tool-accepting, i.e., sized to accept insertion of nasal perforating tool 744, described hereinbelow. Typically, a distal opening 772 of nasal-guide channel 770 faces at least partially in a lateral direction that faces toward lacrimal guide component 50 (typically, this orientation is set (i.e., constrained) by DCR guide 720, such as described hereinabove with reference to FIGS. 5A-D regarding DCR guide 20). Alternatively, distal opening 772 does not face at least partially in the lateral direction. Nasal-guide channel 770 is also shaped so as to define a proximal opening 748.

For some applications, nasal guide component 740 comprises (a) an outer guide element 780 that is shaped so as to define a nasal-shaft-accepting channel therethrough, and (b) a nasal shaft 784 that is slidable through the nasal-shaft-accepting channel, typically before nasal guide component 740 is inserted into the nasal cavity. Nasal shaft 784 is shaped so as to define laterally facing distal opening 42 and nasal-guide channel 770, if provided. For some applications, as shown, nasal shaft 784 comprises a needle, which, for example, may be a Tuohy needle, as is known in the epidural art. As mentioned above, distal opening 772 of nasal-guide channel 770 typically faces at least partially in a lateral direction. For some applications, DCR guide 720 is configured to rotationally orient laterally facing distal opening 42 such that the lateral direction faces toward lacrimal guide component 50. For some applications, nasal guide component 740 further comprises locking mechanism 600, such as described hereinabove with reference to FIGS. 5A-D. For some applications, nasal shaft 784 comprises a proximal handle 728, which may be sized and/or shaped to limit an extent of distal advancement of nasal shaft 784 within the nasal-shaft-accepting channel of outer guide element 780 and additionally positions distal opening 772 to face perforating shaft 30 at all angles.

Reference is now made to FIG. 7, which is a schematic illustration of components of DCR tool 710, in accordance with an application of the present invention. As depicted in FIG. 7, DCR tool 710 further comprises a nasal perforating tool 744, which is sized to pass through nasal-guide channel 770, including proximal and distal openings 748 and 772 thereof. Nasal perforating tool 744 comprises:
a distal nasal-tool perforating tip 758 configured to widen bypass 520 between lacrimal sac 512 and nasal cavity 500, the formation of which is described hereinbelow with reference to FIGS. 8A-C;
a proximal elongate shaft 764; and
a distal flexible portion 766 disposed longitudinally between proximal elongate shaft 764 and distal nasal-tool perforating tip 758.

For some applications, nasal perforating tool 744 comprises a proximal handle 736, which may be sized and/or shaped to limit an extent of advancement of nasal perforating tool 744 within nasal-guide channel 770, e.g., such that distal nasal-tool perforating tip 758 can only protrude from distal opening 772 by up to a certain distance.

For some applications, such as shown in FIG. 7, distal nasal-tool perforating tip 758 is shaped as a drill bit 774. For other applications, distal nasal-tool perforating tip 758 is shaped as a punch (configuration not shown, but similar to punching perforating shaft 230 described hereinabove with reference to FIG. 2B). Further alternatively, distal nasal-tool perforating tip 758 comprises an energy-application perforating tip, which uses energy (e.g., RF, electrical, or laser) to widen the bypass.

Optionally, distal nasal-tool perforating tip 758 (e.g., drill bit 774) has a greatest outer diameter of between 4 and 6 mm, e.g., 2 and 3 mm, such as between 2.5 and 3 mm (e.g., 2.8 mm). Optionally, perforating shaft 30 (e.g., drilling perforating shaft 130) has a greatest outer diameter of between 0.5 and 1.5 mm (e.g., 1 mm). Typically, the greatest outer diameter of distal nasal-tool perforating tip 758 (e.g., drill bit 774) is greater than (e.g., at least 150% of, such as at least 200% of, e.g., at least 250% of) the greatest outer diameter of perforating shaft 30 (e.g., drilling perforating shaft 130).

For some applications and optionally, DCR tool 710 further comprises a guidewire 530 sized to be threadable through at least one of perforating shaft 30, drilling perforating shaft 130, nasal-guide channel 770, including proximal and distal openings 748 and 772 thereof and guidewire-accepting channel 788 of nasal-perforating-tool 744. In some applications, guidewire 530 comprises a stopper 850 e.g., a bead, configured to abut an opening (e.g., opening 855) of lacrimal-guide channel 52 of lacrimal guide component 50. A potential advantage of this configuration is in that it allows for a one-handed tightening of guidewire 530 in the positioning of distal nasal-tool perforating tip 758 as explained in greater detail elsewhere herein.

With regard to nasal perforating tool 744, for some applications, distal flexible portion 766 comprises a coil 768. (Optionally, distal nasal-tool perforating tip 758 comprises a distal tip of coil 768, such that respective portions of a single coil serve both as distal flexible portion 766 and distal nasal-tool perforating tip 758.) Alternatively or additionally, for some applications, distal flexible portion 766 comprises a flexible polymer, metal, or other material, which, for applications in which distal nasal-tool perforating tip 758 comprises drill bit 774, is capable of transmitting sufficient torque for drill bit 774 to widen bypass 520 as described hereinbelow. For example, distal flexible portion 766 may comprise a rope, cable, or other woven elongate member. It is noted that, for some applications, flexible portion 766 may also be encapsulated within a flexible tube such as a Silicone tube.

For some applications, nasal perforating tool 744 is shaped so as to define a nasal-perforating-tool guidewire-accepting channel 788, which is sized to accept insertion of guidewire 530, described hereinbelow with reference to FIGS. 8D-E. Typically, nasal perforating tool 744 is shaped such that nasal-perforating-tool guidewire-accepting channel 788 has a distal opening 798 at a distal end of distal nasal-tool perforating tip 758.

For some applications, support member 62A has a triangular geometry one side or edge 739 of which being disposed between the arced portion of support member 62A and lacrimal guide component 50, providing added support and stability to lacrimal guide component 50 when adjusting the desired angle (a) between lacrimal guide component 50 and perforating shaft 30. For some applications, DCR guide 720 further comprises a locking screw 722, which is arranged to lock first and second arcuate support members 62A and 62B with respect to each other, thereby fixing the desired angle α (alpha) between lacrimal guide component 50 and perforating shaft 30.

For some applications, DCR guide 720 further comprises a knob 724 and/or a knob 726, which are configured to axially lock (a) nasal shaft 784 within the nasal-shaft-accepting channel of outer guide element 780 and/or (b) nasal perforating tool 744 within nasal-guide channel 770.

Reference is now made to FIGS. 8A-G, which are schematic illustrations of a method for performing DCR and FIG. 8H, which is a flowchart illustrating a method of performing dacryocystorhinostomy (DCR), in accordance with an application of the present invention. The method may implement any of the techniques of method 400, described hereinabove with reference to FIGS. 3 and 4A-I, mutatis mutandis.

As shown in FIG. 8A, at nasal guide insertion steps 802 and 804, nasal guide component 740 of DCR guide 720 is inserted into nasal cavity 500 and perforating shaft 30 (e.g., perforating shaft 130, as shown) is advanced through lacrimal-guide channel 52 of lacrimal guide component 50 of DCR guide 720.

As shown in FIG. 8B, at perforation step 806, perforating shaft 30 (e.g., perforating shaft 130, as shown) is advanced through and lacrimal passageway 510 and into lacrimal sac 512. A bypass 520 is formed between lacrimal sac 512 and nasal cavity 500 by advancing distal perforating-shaft perforating tip 32 through lateral side 522 of lacrimal sac 512, lacrimal bone 524, and nasal mucosa 526. In operation, perforating shaft 30 (e.g., perforating shaft 130, as shown) is advanced through distal opening 772 of nasal-guide channel 770 until perforating tip 32 perforating shaft 30 engages an internal wall 875 of the nasal-shaft-accepting channel located opposite to the laterally facing distal opening of the nasal guide component. A potential advantage in this configuration is in that internal wall 875 of the nasal-shaft-accepting channel blocks further advancing of distal perforating-shaft perforating tip 32 of nasal guide component 740. This contact prevents over-advancement distal perforating-shaft perforating tip 32, which might otherwise perforate tissue across nasal cavity 500, which is generally no more than several millimeters beyond bypass 520.

For some applications, the surgeon may form bypass 520 using a perforating tool that is not a component of DCR tool 710 (e.g., either mechanically or using energy, e.g., laser energy), either using or not using DCR tool 710. Arcuate support members 62A and 62B and lacrimal guide component 50 are oriented such that any extension or retraction of arcuate support members 62A and 62B in relation to each other, varies angle (a) between respective central longitudinal axes of nasal guide component 740 and perforating shaft 30, while maintaining a fixed point of intersection of the axes at distal opening 772 of nasal-guide channel 770.

In operation, once angle (a) between respective central longitudinal axes of nasal guide component 740 and perforating shaft 30 is adjusted, locking screw 722 is activated (e.g., rotated) and locks first and second arcuate support members 62A and 62B with respect to each other, thereby fixing the desired angle α (alpha) between lacrimal guide component 50 and perforating shaft 30 and ensuring engagement of perforating shaft 30 with internal wall 875 of the nasal-shaft-accepting channel located opposite to the laterally facing distal opening of the nasal guide component.

For some applications, as shown in FIG. 8C, at guidewire placement steps 808 and 810, once positioned inside lacrimal-guide channel 52 of lacrimal guide component 50 and bypass 520, drilling perforating shaft 130 is locked in place by a locking mechanism 650. In some applications, locking mechanism 650 is a rotatable locking mechanism similar to that of a drill chuck. A guidewire 530 is then placed such that guidewire 530 passes through lacrimal passageway 510, bypass 520, and at least a portion of nasal-guide channel 770 (labeled in FIGS. 6A-B and 7). Typically, guidewire 530 is placed such that guidewire 530 passes through drilling perforating shaft 130 left in lacrimal passageway 510 after forming bypass 520, bypass 520, the entire nasal-guide channel 770, and out of proximal opening 748 of nasal-guide channel 770. Typically, distal opening 772 of nasal-guide channel 770 faces at least partially in the lateral direction that faces toward lacrimal guide component 50, such as described hereinabove with reference to FIGS. 6A-B and 7.

Further at guidewire insertion step 810, guidewire 530 is advanced through lacrimal guidewire-accepting channel 94 of perforating shaft 30 while perforating shaft 30 is disposed passing through lacrimal passageway 510, such that guidewire 530 passes through lacrimal passageway 510. Typically, guidewire 530 is advanced through lacrimal guidewire-accepting channel 94 while (a) perforating shaft 30 is disposed passing through lacrimal passageway 510 and bypass 520, and (b) distal perforating-shaft perforating tip 32 has at least entered distal opening 772 of nasal-guide channel 770 of nasal guide component 740.

As shown in FIGS. 8D and 8E, at a nasal perforating tool step 812, nasal perforating tool 744 (e. g., dilator tool) is driven over a distal end 820 of guidewire 530 extending from proximal handle 728 of DCR guide 710 such that distal end 820 extends out of proximal handle 736 and proximal handle 736 of nasal perforating tool 744 abuts handle 728. Nasal perforating tool 744 is then locked in place (e.g., by rotating handle 728). as shown in FIGS. 8E and 8F, at nasal perforating tool step 814, guidewire 530 is tightened (FIG. 8E) forcing nasal perforating tool 744 distal nasal-tool perforating tip 758 out of distal opening 772 of nasal shaft 784 into contact with a medial opening of bypass 520 (FIG. 8F), and, using distal nasal-tool perforating tip 758, bypass 520 between lacrimal sac 512 and at step 816 nasal cavity 500 is widened.

In some applications, distal nasal-tool perforating tip 758 is not advanced or urged out of laterally facing distal opening 772. Distal nasal-tool perforating tip 758 is directed out of opening 772 by virtue of tension alone placed on guidewire 530 being pulled out of proximal handle 736 nasal perforating tool 744. Pulling on guidewire 530 not only places tension on it, but also shortens guidewire 530 length between proximal handle 736 and stopper 850 thus flexing distal nasal-tool perforating tip 758 out of distal laterally facing opening 772. Hence distal nasal-tool perforating tip 758 flexes passively and conforms to the shortening and therefore change of the angle of guidewire 530 in the section between distal laterally facing opening 772 of nasal-guide channel 770 and distal perforating-shaft perforating tip 32.

Widening bypass 520 using nasal perforating tool 744 may be particularly useful for applications in which forming a wider bypass 520 using perforating shaft 30 is difficult. For example, the small diameter of lacrimal passageway 510 may limit the maximum diameter of perforating shaft 30.

At step 818, and as shown in FIG. 8G, the tools are removed from the subject's body, leaving guidewire 530 in place.

For some applications, the method for performing DCR comprises a support step 820, as described hereinabove with reference to FIGS. 4H-I.

As described above, for some applications nasal-guide channel 770 is configured to be both nasal-perforating-tool-accepting and guidewire-accepting, and nasal perforating tool 744 is shaped so as to define nasal-perforating-tool guidewire-accepting channel 788. In these applications, guidewire placement step 408 is performed. Subsequently, at the nasal perforating tool step described hereinabove with reference to FIG. 8D, in order to advance nasal perforating tool 744 within nasal-guide channel 770 until distal nasal-tool perforating tip 758 exits the distal opening of distal nasal-tool perforating tip 758: (a) guidewire 530 is threaded into nasal-perforating-tool guidewire-accepting channel 788, and (b) nasal perforating tool 744 is advanced over guidewire 530.

For some applications, at the bypass widening step described above with reference to FIG. 8E, nasal perforating tool 744 is advanced within nasal-guide channel 770 until distal nasal-tool perforating tip 758 exits distal opening 772 of distal nasal-tool perforating tip 758 as a central longitudinal axis 776 of distal nasal-tool perforating tip 758 flexes by at least 15 degrees (e.g., at least 30 degrees, e.g., at least 40 degrees, such as at least 45 degrees, e.g., at least 60 degrees, such as about 75 to 90 degrees as schematically illustrated in FIGS. 8E and 8F) with respect to a central longitudinal axis 778 of nasal-guide channel 770.

For some applications, nasal-guide channel 770 is shaped so as to define a distal surface 786 that faces partially in a proximal direction and partially in the lateral direction toward lacrimal guide component 50 so as to direct distal nasal-tool perforating tip 758 out of distal opening 772 of nasal-guide channel 770, thereby flexing distal flexible portion 766, when nasal perforating tool 744 is advanced distally through nasal-guide channel 770. (Optional characteristics of distal surface 786 are described hereinbelow.)

Reference is made to FIGS. 6A-B. For some applications, distal flexible portion 766 is sufficiently flexible to allow flexing of central longitudinal axis 776 (FIG. 8E) of distal nasal-tool perforating tip 758 by at least 15 degrees (e.g., at least 30 degrees, e.g., at least 40 degrees, such as at least 45 degrees, e.g., at least 60 degrees, such as about 75 to 90 degrees as schematically illustrated in FIG. 8E) with respect to central longitudinal axis 778 of nasal-guide channel 770, without causing plastic deformation of distal flexible portion 766 (optionally, at 37 degrees C.).

Reference is made to FIGS. 6A-B, 7, and 8E. For some applications, distal flexible portion 766 is sufficiently flexible to allow flexing of central longitudinal axis 776 of distal nasal-tool perforating tip 758 by at least 15 degrees (e.g., at least 30 degrees, e.g., at least 40 degrees, such as at least 45 degrees, e.g., at least 60 degrees, such as about 75 to 90 degrees as schematically illustrated in FIG. 8E) with respect to central longitudinal axis 778 of nasal-guide channel 770 when distal nasal-tool perforating tip 758 is advanced out of distal opening 772 of nasal-guide channel 770 while the nasal guide component is within the nasal cavity.

Reference is still made to FIGS. 6A-B, 7, and 8E. As mentioned above, for some applications distal opening 772 of nasal-guide channel 770 faces at least partially in a lateral direction that faces toward lacrimal guide component 50. For some of these applications, nasal-guide channel 770 is shaped so as to define distal surface 786 that faces partially in a proximal direction and partially in the lateral direction toward lacrimal guide component 50 so as to direct distal nasal-tool perforating tip 758 out of distal opening 772 of nasal-guide channel 770, thereby flexing distal flexible portion 766, when nasal perforating tool 744 is advanced distally through nasal-guide channel 770.

For some applications, distal surface 786 includes at least one curved surface, which may have a single radius of curvature or a plurality of different radii of curvature. For example, each of the one or more radii of curvature may be between 2.67 and 16.1 mm, or may have other values. Alternatively or additionally, for some applications, distal surface 786 includes at least one planar surface; distal surface 786 may be partially or entirely planar.

Reference is still made to FIGS. 6A-B, 7, and 8E. As mentioned above, for some applications nasal perforating tool 744 is shaped so as to define nasal-perforating-tool guidewire-accepting channel 788 having distal opening 798 at the distal end of distal nasal-tool perforating tip 758. For some of these applications, distal flexible portion 766 is sufficiently flexible to allow flexing of central longitudinal axis 776 of distal nasal-tool perforating tip 758 by at least 15 degrees (e.g., at least 30 degrees, e.g., at least 40 degrees, such as at least 45 degrees, e.g., at least 60 degrees, such as about 75 to 90 degrees as schematically illustrated in FIG. 8E) with respect to central longitudinal axis 778 of nasal-guide channel 770 when distal nasal-tool perforating tip 758 is advanced over the guidewire and out of distal opening 772 of nasal-guide channel 770 while the nasal guide component is within the nasal cavity and the guidewire is within nasal-perforating-tool guidewire-accepting channel 788.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. Apparatus for performing dacryocystorhinostomy (DCR), the apparatus comprising a dacryocystorhinostomy (DCR) tool, which comprises:
a perforating shaft having a distal perforating-shaft perforating tip configured to form a bypass between a lacrimal sac and a nasal cavity through a lateral side of the lacrimal sac, a lacrimal bone, and nasal mucosa; and
a DCR guide, which comprises:
a nasal guide component, which is configured to be inserted into the nasal cavity and has a nasal-guide channel having a proximal opening and an at least partially laterally facing distal opening; and
a lacrimal guide component, which is shaped so as to define a lacrimal-guide channel that is configured to orient the distal perforating-shaft perforating tip with respect to the DCR guide during advancing of the distal perforating-shaft perforating tip through a lacrimal passageway and into the lacrimal sac, until the distal perforating-shaft perforating tip at least crosses the laterally facing distal opening of the nasal guide component,
wherein the DCR guide is configured to constrain the laterally facing distal opening of the nasal guide component to fall in a path of advancement of the distal perforating-shaft perforating tip.

2. The apparatus according to claim 1, wherein the DCR guide is configured to set a desired angle between respective central longitudinal axes of the nasal guide component and the laterally facing distal opening.

3. The apparatus according to claim 2, wherein the DCR guide is shaped so as to define an arcuate portion that is configured to allow relative movement between the nasal guide component and the lacrimal guide component to set the desired angle.

4. The apparatus according to claim 1, wherein the distal perforating-shaft perforating tip is shaped as a drill bit.

5. The apparatus according to of claim 1, wherein the nasal-guide channel is configured to be guidewire-accepting.

6. The apparatus according to claim 5, wherein the distal opening of the nasal-guide channel faces at least partially in toward the lacrimal guide component.

7. The apparatus according to claim 5, wherein the nasal guide component comprises (a) an outer guide element that is shaped so as to define a nasal-shaft-accepting channel therethrough, and (b) a nasal shaft that is slidable through the nasal-shaft-accepting channel, and wherein the nasal shaft is shaped so as to define a distal guide tip and the nasal-guide channel, and
wherein when the outer guide element and the nasal shaft are coupled together, a distal opening of the nasal-guide channel is constrained by the outer guide element and the nasal shaft to face at least partially in a lateral direction that faces toward the lacrimal guide component.

8. The apparatus according to claim 7, wherein the nasal guide component comprises a locking mechanism, which is configured to lock the nasal shaft with respect to the nasal-shaft-accepting channel, thereby maintaining the distal opening of the nasal-guide channel facing at least partially in the lateral direction.

9. The apparatus according to claim 5, wherein the perforating shaft, including the distal perforating-shaft perforating tip, is shaped so as to define a lacrimal guidewire-accepting channel.

10. The apparatus according to claim 5, wherein the DCR tool further comprises a dilator, which is configured to be advanced through the nasal-shaft-accepting channel and laterally facing distal opening and into the bypass, and to dilate the bypass.

11. (canceled)

12. The apparatus according to of claim 1, wherein the nasal guide component is shaped so as to define a nasal-guide channel, which is shaped so as to define proximal and distal openings and configured to be nasal-perforating-tool-accepting, and wherein the DCR tool further comprises a nasal perforating tool, which is sized to pass through the nasal-guide channel, including the proximal and the distal openings thereof, and which comprises:
a distal nasal-tool perforating tip configured to widen the bypass between the lacrimal sac and the nasal cavity;
a proximal elongate shaft; and
a distal flexible portion disposed longitudinally between the proximal elongate shaft and the distal nasal-tool perforating tip.

13. The apparatus according to claim 12, wherein the distal nasal-tool perforating tip is shaped as a drill bit.

14. The apparatus according to claim 12, wherein the nasal perforating tool is shaped so as to define a nasal-perforating-tool guidewire-accepting channel.

15. The apparatus according to claim 14, wherein the nasal perforating tool is shaped such that the nasal-perforating-tool guidewire-accepting channel has a distal opening at a distal end of the distal nasal-tool perforating tip.

16. The apparatus according to claim 15, wherein the dacryocystorhinostomy (DCR) tool device is for use with a guidewire, and wherein the distal flexible portion is sufficiently flexible to allow flexing of a central longitudinal axis of the distal nasal-tool perforating tip with respect to a central longitudinal axis of the nasal-guide channel when the distal nasal-tool perforating tip is advanced over the guidewire and out of the distal opening of the nasal-guide channel while the nasal guide component is within the nasal cavity and the guidewire is within the nasal-perforating-tool guidewire-accepting channel.

17. The apparatus according to claim 1, wherein the lacrimal guide component comprises a locking mechanism, which is configured to lock the perforating-shaft with respect to the lacrimal-guide channel of the lacrimal guide component.

18. A method of performing dacryocystorhinostomy (DCR), the method comprising:
inserting a nasal guide component of a dacryocystorhinostomy (DCR) guide into a nasal cavity of a patient's body the guide having a laterally facing distal opening;
advancing a perforating shaft through a lacrimal-guide channel of a lacrimal guide component of the DCR guide and a lacrimal passageway and into a lacrimal sac, the lacrimal passageway including a lacrimal punctum, a lacrimal canal, and a common canaliculus; and
forming a bypass between the lacrimal sac and the nasal cavity by advancing a distal perforating-shaft perforating tip through a lateral side of the lacrimal sac, a lacrimal bone, and nasal mucosa, until the distal perforating-shaft perforating tip at least enters the laterally facing distal opening, wherein the DCR guide constrains the laterally facing distal opening of the nasal guide component to fall in a path of advancement of the distal perforating-shaft perforating tip.

19. The method according to claim 18, wherein inserting the nasal guide component and the lacrimal guide component comprises using the DCR guide to set a desired angle between respective central longitudinal axes of the nasal guide component and the perforating shaft.

20. The method according to claim 19, wherein setting the desired angle comprises using an arcuate portion of the DCR guide that allows relative movement between central longitudinal axes of the nasal guide component and the lacrimal guide component, while maintaining a fixed point of intersection of the axes at the laterally facing distal opening.

21. The method according to claim 20,
